SEQUENCE LISTING (37 sequences)

SEQ ID NO: 1 - DNA, 17 bp, Artificial Sequence
  primer
tcggagcctgcggagat

SEQ ID NO: 2 - DNA, 22 bp, Artificial Sequence
  primer
gtcctccctggctgttatcttc

SEQ ID NO: 3 - DNA, 20 bp, Artificial Sequence
  primer
aaaggagaatgggacctctg

SEQ ID NO: 4 - DNA, 22 bp, Artificial Sequence
  primer
cgctgggacataaatctttgag

SEQ ID NO: 5 - DNA, 20 bp, Artificial Sequence
  primer
ttgctaaaggcttccgagac

SEQ ID NO: 6 - DNA, 20 bp, Artificial Sequence
  primer
aggtggaggaacttgcattg

SEQ ID NO: 7 - DNA, 20 bp, Artificial Sequence
  primer
acagcgttctgcactcacgt

SEQ ID NO: 8 - DNA, 22 bp, Artificial Sequence
  primer
catgtcatcactgccccgaaat

SEQ ID NO: 9 - DNA, 20 bp, Artificial Sequence
  primer
tctatccacgtgcctacagc

SEQ ID NO: 10 - DNA, 20 bp, Artificial Sequence
  primer
gttgtccgcctctgtcttct

SEQ ID NO: 11 - DNA, 20 bp, Artificial Sequence
  primer
tgatgaagcaactccagcag

SEQ ID NO: 12 - DNA, 20 bp, Artificial Sequence
  primer
ggactggctaccatgctgtt

SEQ ID NO: 13 - DNA, 20 bp, Artificial Sequence
  primer
gtctttgtttggggcaagag

SEQ ID NO: 14 - DNA, 20 bp, Artificial Sequence
  primer
gattggatggcagtttggag

SEQ ID NO: 15 - DNA, 20 bp, Artificial Sequence
  primer
aatcaagcagaggcgaagtc

SEQ ID NO: 16 - DNA, 20 bp, Artificial Sequence
  primer
tctcgtcaaaaagcctctcc

SEQ ID NO: 17 - DNA, 19 bp, Artificial Sequence
  primer
gtgctggcccttcaactac

SEQ ID NO: 18 - DNA, 19 bp, Artificial Sequence
  primer
gcagcttctccaccttagc

SEQ ID NO: 19 - DNA, 25 bp, Artificial Sequence
  primer
gtgtttaagggcttttgaaaactgc

SEQ ID NO: 20 - DNA, 26 bp, Artificial Sequence
  primer
cattccaccaacatttattaagcagc

SEQ ID NO: 21 - DNA, 24 bp, Artificial Sequence
  primer
acagtccaggcttagaaggatgtc

SEQ ID NO: 22 - DNA, 26 bp, Artificial Sequence
  primer
acagcattaggcaccaagggtaggag

SEQ ID NO: 23 - DNA, 24 bp, Artificial Sequence
  primer
gtccatgctgtactacctgggact

SEQ ID NO: 24 - DNA, 24 bp, Artificial Sequence
  primer
gtagcggttctcctctctagcctg

SEQ ID NO: 25 - DNA, 21 bp, Artificial Sequence
  primer
ccacctccaacaacacgttct

SEQ ID NO: 26 - DNA, 20 bp, Artificial Sequence
  primer
taaggaaacaggctcccgaa

SEQ ID NO: 27 - DNA, 20 bp, Artificial Sequence
  primer
tgtcccccatcaagcctgtt

SEQ ID NO: 28 - DNA, 21 bp, Artificial Sequence
  primer
atggcctccagaatgcgtatg

SEQ ID NO: 29 - DNA, 22 bp, Artificial Sequence
  primer
accaattttaccctggaacaac

SEQ ID NO: 30 - DNA, 21 bp, Artificial Sequence
  primer
tcgattttgaaaccaaacctg

SEQ ID NO: 31 - DNA, 21 bp, Artificial Sequence
  primer
ctaacatcaaatggggtgagg

SEQ ID NO: 32 - DNA, 20 bp, Artificial Sequence
  primer
cggagatgatgacccttttg

SEQ ID NO: 33 - DNA, 34 bp, Artificial Sequence
  primer
acccgcagtctttccaatataccatttcagccca

SEQ ID NO: 34 - DNA, 28 bp, Artificial Sequence
  primer
aagtcgtgctgcttcatgtggtcggggt

SEQ ID NO: 35 - DNA, 24 bp, Artificial Sequence
  primer
tggcggaatggtgagcaagggcga

SEQ ID NO: 36 - DNA, 26 bp, Artificial Sequence
  primer
atgggagataagggggtgggaggaga

SEQ ID NO: 37 - DNA, 20 bp, Homo sapiens
ggcgttggcgtcacagaccc

(19) United States
(12) Patent Application Publication
(10) Pub. No.: US 2023/0000924 A1
(43) Pub. Date: Jan. 5, 2023

(54) COMPOSITIONS AND METHODS FOR GENERATION OF SINOATRIAL NODE-LIKE CELLS AND THEIR USE IN DRUG DISCOVERY

(71) Applicant: Cornell University, Ithaca, NY (US)

(72) Inventors: Shuibing CHEN, Pelham, NY (US); Todd EVANS, Pelham, NY (US); Zaniar GHAZIZADEH, New Haven, CT (US)

(21) Appl. No.: 17/781,265

(22) PCT Filed: Dec. 2, 2020
(86) PCT No.: PCT/US2020/062919

§ 371 Date: May 31, 2022

Related U.S. Application Data
(60) Provisional application No. 62/942,505, filed on Dec. 2, 2019.

(51) Int. Cl. A61K 35/34; A61K 31/203; A61K 31/404; A61K 31/4439; A61K 31/519; A61K 31/575; C12N 5/077; G01N 33/50

(52) U.S. Cl. CPC A61K 35/34; A61K 31/203; A61K 31/404; A61K 31/4439; A61K 31/519; A61K 31/575; C12N 5/0657; G01N 33/5023; C12N 2510/00; C12N 2501/155; C12N 2501/11; C12N 2501/16; C12N 2506/45; C12N 2503/02; C12N 2501/727; C12N 2506/02; C12N 2501/415

(57) ABSTRACT

Provided are methods for producing population of cells enriched for cells exhibiting sinoatrial node like characteristics. The cells can be produced from human pluripotent cells. Also provided are methods for using the SAN-like cells for identifying agents that can mitigate drug-induced cardiac toxicity. Also provided is a method for mitigating drug induced cardiotoxicity comprising administering to a subject an effective amount of physcion or a derivative thereof.

### CROSS-REFERENCE TO RELATED APPLICATIONS

This application claims priority to U.S. Provisional patent application No. 62/942,505, filed on Dec. 2, 2019, the disclosure of which is incorporated herein by reference.

### BACKGROUND OF THE DISCLOSURE

The sinoatrial node (SAN) is the primary pacemaker of the heart. The human SAN is poorly understood due to limited primary tissue access and lack of robust in vitro derivation methods. There is a need for methods to isolate cells with SAN characteristics, or to generate cells from stem cells that exhibit SAN characteristics so that effects of various therapeutics on SAN function can be investigated.

Previous efforts have focused on in vivo direct conversion or an NKX2.5 negative selection approach to obtain nodal like cells. However, due to lack of a purification method, the studies were limited in characterization and validation of nodal identity for the cells at cellular and molecular levels (Kapoor et al., Nat Biotechnol 31, 54-62 (2013); Protze et al., Nat Biotechnol 35, 56-68 (2017), and therefore use of such cells for drug discovery is limited.

### SUMMARY OF THE DISCLOSURE

The present disclosure provides compositions and methods for generating cells with sinoatrial node (SAN) characteristics from pluripotent cells, such as human pluripotent stem cells. The method is based on a strategy using a dual SHOX2:GFP; MYH6:mCherry knock-in reporter line to generate and purify human pluripotent stem cell-derived SAN cells (hPSC-SAN), displaying molecular and electrophysiological characteristics of bona-fide nodal cells.

The method was developed by using the steps comprising i) providing a population of pluripotent stem cells, such as human pluripotent cells, ii) introducing into the cells, a reporter for SHOX2 expression, iii) incubating the cells in a medium comprising retinoic acid, an EGFR/FGFR inhibitor, Wnt inhibitor, and optionally, a STAT3 inhibitor, iv) optionally, further incubating the cells in a medium further comprising HDAC inhibitor, and v) sorting for cells that are positive for the reporter for SHOX2. This process generates SAN-like cells. These cells (SAN-like cells) display SA node like biochemical and electrophysiological characteristics. In an embodiment, the method uses media further comprising Activin Receptor inhibitor. The cells may be used for implantation or for screening (such as in a high throughput screening) to identify agents that can affect the functioning of SAN cells.

In an embodiment, SAN-like cells may be generated by incubating PSCs (such as hPSCs) with a medium comprising a GSK inhibitor, and one or more members of the TGF-β superfamily of proteins (member of TGF-β SFP), for a sufficient period of time to generate pre-cardiac mesoderm cells. The pre-cardiac mesoderm cells are incubated in a medium comprising retinoic acid signaling activator, Wnt inhibitor, Activin receptor (ALK) inhibitor, member of TGF-β SFP and/or EGFR inhibitor, and optionally, STAT3 inhibitor, for a period of time sufficient to generate pacemaker progenitor cells. The pacemaker progenitor cells are then incubated in a medium comprising a GSK inhibitor, and EGFR inhibitor and optionally, a HDAC inhibitor for a sufficient period of time to generate SA-node like cells. In an embodiment, the pacemaker progenitor cells are first incubated for a period of time in a medium comprising GSK inhibitor and a EGFR inhibitor, and after about 3-7 days, the medium may additionally comprise a HDAC inhibitor. After this, the cells can then be continued in culture in a serum-free, growth factor fee medium and by day 30 (total time in culture) exhibit pacemaker markers and electrophysiological properties.

In an embodiment, the SAN-like cells generated by the present methods can be used to evaluate cell type specific toxicity upon treatment with therapeutics for various diseased conditions. For example, cancer treatments cause cardiovascular toxicities including arrhythmias and cardiomyopathy. Doxorubicin (DOXO) remains a key component of regimens to treat a wide spectrum of solid tumors and leukemia. DOXO-induced cardiomyopathy ranges from 10-20% and sub-clinical systolic dysfunction is estimated at 40% of treated patients. The SAN-like cells generated by the present methods can be used to evaluate therapeutic candidates for their cardiac toxicity effects, and also to evaluate candidate agents that could mitigate cardiac toxicity associated with therapeutics like anthracyclines, including doxorubicin.

Further, using this technique, we discovered 3 new genetic loci associated with increased sensitivity to DOXO-induced hPSC-SAN death. Genetic variants in these loci were associated with significantly higher early arrhythmia risk in patients receiving DOXO, confirmed by an unbiased PheWAS analysis. The in vitro DOXO assay enables an unbiased drug screening platform and identification of candidate therapeutic that can partially block DOXO-mediated cardiac toxicity.

### BRIEF DESCRIPTION OF THE FIGURES

FIG. 1. Generation of human SAN-like cells from human pluripotent stem cells. a, Schematic representation of the differentiation protocols; b,c,d Quantitative PCR (b), live fluorescence images (c) and FACS plots (d) of the day 30 cells derived from the SHOX2:NLS-eGFP;MYH6:mCherry H9 line using the corresponding differentiation protocols (n=3 for protocol 1,2 and =6 for protocols 3-8). For quantitative PCR data, fold changes were normalized to protocol #1. *p<0.05 **p<0.01, ***p<0.001, ANOVA. Data are mean±s.e.m; e, Quantitative PCR analysis for SHOX2 transcriptional expression of GFP⁺ cells purified after sorting. n=3 independent experiments. ***p<0.001, t-test. Data are mean±s.e.m and normalized to GFP-negative population; f, Schematic and results of the epigenetic library chemical screen; g, Immunofluorescence image and FACS plot of day 30 cells derived using protocol described in (f), Scale bar=100 μM; h, i, Hierarchical clustering (h) and heatmap (i) of transcriptional profiling in GFP⁺ cells and mCherry⁺ cells purified as described in (f). Murine sinoatrial and atrial tissues (GSE65658) were used as controls. RtA: murine right atrial sample SAN: murine sinoatrial node sample.

FIG. 2. Functional characterization of hPSC-SAN cells. a, Immunofluorescent co-staining of GFP with pacemaker-associated transcription factors (ISL1, TBX5 and NKX2.5) and ion channels (HCN4, Cav3.1, Cav1.3 and Cx30.2) in cells differentiated to day 30 or day 60. Scale bar=200 μM. b, qRT-PCR analysis using samples from GFP⁺ (G+) or mCherry⁺ (Ch+) cells at day 25 or day 45. n=3 independent experiments. Fold-change was normalized to undifferentiated hESC. c, Schematic for co-culture of hESC-derived ANs expressing eYFP-channelrhodopsin-2 with either hPSC-SAN or hESC-V; d, Immunofluorescence (left) and electron microscopy images (right) of hESC-AN and hPSC-SAN after 12 weeks of co-culture. Scale bar: 100 μM and 1 μM, respectively; e, Basal release of catecholamine was analyzed using an ELISA kit for AN alone, AN+hES-SAN and AN+hES-V conditions after 8 weeks of co-culture. n=3 independent experiments. f, Representative image of averaged action potentials recorded from an individual hPSC-SAN (left) and distribution (right) of dV/dtₘₐₓ, APD30 and APD90 recorded used day 30 cells; g, Quantitative analysis of day 30 hPSC-SAN function in response to 1 μM ivabradine. Fold change in calcium transient amplitude (deltaF/F0) of hPSC-SAN or hESC-V cells before and after treatment with ivabradine (n=22 for hESC-SAN and =17 for hESC-V). *p<0.05 **p<0.01. t-test. Data are mean±s.e.m.

FIG. 3. Single-cell RNA sequencing analysis of differentiated hESC-SAN and hESC-V cells. a, t-SNE plots colored according to the cell populations. b, Heatmap based on structural protein expression showing the clustering of the high-SHOX2, mixed and no-SHOX2 cells. c, PCA plot of high-SHOX2 and no-SHOX2 cells; d, Jitter plots based on structural protein expression of high-SHOX2 and no-SHOX2 cells. e, f, Heatmap (e) and jitter plots (f) based on expression of key transcription factors and ion channels in high-SHOX2 and no-SHOX2 cells. g, h, Joy plots for the expression of HCN4, ISL1, TBX3, TBX5 and TBX18 (g) and jitter plots for the expression of SHOX2, CDON, NR2F1 and VSNL1, (h) in the high-SHOX2 and no-SHOX2 cells.

FIG. 4. Pharmacogenetics and drug discovery for doxorubicin-mediated acute cell toxicity using hPSC-SAN cells. a, Relative cell toxicity of hESC-SAN and hESC-V cells upon DOXO treatment based on immunostaining of antibody against cleaved-caspase3. The fold change was calculated by dividing the percentage of cleaved caspase-3⁺ cells in GFP⁺ or mCherry⁺ cells in the DOXO-treated condition by the percentage of cleaved caspase-3⁺ cells in GFP⁺ or mCherry⁺ cells in the DMSO-treated condition (n=10); b, Sensitivity of day 30 hiPSC-SAN cells derived from 48 hiPSC lines upon DOXO treatment. 48 hiPSC lines were differentiated toward SAN-like cells and split into two equal samples, which were treated with 0.16 μM doxorubicin or DMSO for 3 days. The fold change was calculated by dividing the percentage of cleaved caspase-3⁺ cells in ISL1⁺ cells in the DOXO-treated condition by the percentage of cleaved caspase-3⁺ cells in ISL1⁺ cells in the DMSO-treated condition. Each dot represents the average fold change for cell toxicity in hPSC-SAN cells derived from an individual iPSC line; c, The hPSC-SAN cells derived from different iPSC lines were grouped based on the risk allele of rs1056892, rs2229774, rs885004 or none of these variants; d, Manhattan plot showing the genetic loci associated with DOXO-induced SAN cell toxicity; e, The genetic loci identified from the iPSC screen that are confirmed with significant increased risk in the patient cohort. f, The panel represents phenotypes tested for association with SNPs related to LIG4 and ANO2. Each point represents the −log 10 (p) of a SNP-phenotype association tested with PheWAS; g, Dose-response curve of physcion to rescue DOXO-induced cell toxicity. Data were normalized to non-DOXO treated cells (n=4). h, Quantification of cell toxicity on hPSC-SAN cells derived from sensitive iPSC lines after co-treatment with DOXO and either 10 μM physcion (n=3) or DMSO (n=4). *p<0.05 **p<0.01. t-test. Data are mean±s.e.m. Data were normalized to non-DOXO treated cells.

FIG. 5. Optimization of the protocol for the derivation of SAN-like cells from hPSCs a, Schematic representation of the murine differentiation protocol and qRT-PCR analysis for the expression levels of TBX3, TBX18 and SHOX2 following induction of GATA4, GATA5 or GATA6 at day 4. Fold change was normalized to level using the iGATA6 line; b, Heatmap illustration of the qPCR results of transcription-screen for TBX3, TBX18 and SHOX2; hit compounds (EGF inhibitors) from plates 1 and 3 are circled. c, The activity of tyrphostin AG490 and 2NP on the expression of TBX3, TBX18 and SHOX2; d, The activity of cucurbitacin (STAT3 inhibitor) on expression of MYH6; e, qRT-PCR analysis for TBX3, TBX18 and SHOX2 in S1+RA (left) and S1+cucurbitacin (right); f, The percentage of SHOX2:GFP⁺ cells treated with different EGF inhibitors (left) and dose-response curve for effects of Tyrphostin AG490 on the percentage of SHOX2:GFP⁺ cells. Fold changes were normalized to DMSO-treated group unless stated otherwise.

FIG. 6. Generation of a dual-reporter system for characterization of SAN-like cells generated from hESCs a, Schematic representation of dual reporter line; b, Flow cytometry of hESC-derived SAN-like cells for SHOX2:GFP, co-stained for ISL1 or HCN4; c, Schematic representation of the epigenetic library screen; d, Representative flow cytometry analysis plot of hESC-derived SAN cells.

FIG. 7. Transcriptome profiling of hPSC-SAN cells. a, Heatmap analysis of metabolic and signaling pathways; b, gene set enrichment analysis in purified GFP⁺ cells and GFP⁻mCherry⁺ cells. Murine sinoatrial and atrial tissues (GSE65658) were used as controls. RtA: murine right atrial sample SAN: murine sinoatrial node sample.

FIG. 8. Immunofluorescent images of mCherry and DAPI related to FIG. 2a. Scale bar=200 μM.

FIG. 9. Assessment of the effects of Doxorubicin on hPSC-SAN cells. a, Schematic representation of experimental design; b, Immunofluorescence images of hPSC-SAN and hPSC-V cells treated with different doses of DOXO. Scale bar: Upper panels=200 μM (left panel), 75 μM (middle panel) and 100 μM (right panel). Lower panels=200 μM. c, Immunofluorescence images of DOXO toxicity on hPSC-SAN cells derived from non-sensitive iPSC line (iPS3) or sensitive iPSC line (iPS 33) by co-staining for cleaved-caspase3 and ISL1. Scale bar=300 μM.

FIG. 10. Association of doxorubicin sensitivity candidate loci with PheWAS phenotype. Strength of the association is plotted along the y-axis as −log(p value).

FIG. 11. Chemical screening for compounds that rescue the DOXO toxicity of hPSC-SAN cells. a, Primary screening result. Day 35 cells were treated for 3 days with DOXO and small molecules. CTL: no DOXO treated condition; b, Immunofluorescence images of hPSC-SAN cells treated with DMSO (upper panel) or 10 μM physcion (lower panel) in the presence of 0.16 μM DOXO. Scale bar=200 μM. c-e, Immunofluorescence images of DOXO-treated SAN-like cells derived from iPS7 (c), iPS33 (d) and iPS37 (e) treated with DMSO (upper panel) or 10 μM physcion (lower panel). Scale bar=300 μM.

FIG. 12. Doxorubicin causes cell death of murine SAN cells. Immunofluorescence of intact atria isolated from Doxorubicin (two IP injections of 10 mg/kg at 3 day intervals) treated mice euthanized 9 days after initial treatment. Cell death (caspase 3, green) was analyzed in either myocardial (cTroponin, blue) or SAN tissue (HCN4, red). Atria of doxorubicin treated mice exhibited cell death localized to the SAN.

FIG. 13. Doxorubicin causes abnormal arrhythmia in the murine heart. Contractions of intact atria isolated from vehicle (left panels) or Doxorubicin (right panels) treated mice (30 sec recordings;) x axis=contraction (au); y axis=time (ms). Control atria exhibited steady, rhythmic contractions. Doxorubicin treated atria exhibited irregular beating marked by intermittent pauses between contractions, consistent with SAN dysfunction. Quantification of contraction rhythmicity of hearts was calculated by the coefficient of variance (COV)=std dev of peak to peak contraction time/average peak to peak contraction time. As can be seen in the graphs, atria of control mice exhibited rhythmic contractions that exhibited minimal variance in the time between contractions. By contrast, atria of mice treated with Doxorubicin exhibited highly irregular contractions, with a significantly higher variance in the time between contractions.

FIG. 14. Vehicle-injected mice do not show cell death in murine SAN cells. Control vehicle-injected (DMSO) mice did not exhibit cell death (caspase 3, green) in either myocardial (cTroponin, blue) or SAN tissue (HCN4, red).

FIG. 15. Doxorubicin causes abnormal arrhythmia in explanted murine hearts ex vivo. Quantification of contraction rhythmicity of explants was calculated by the COV as described above. Atria explants of control mice (left panels) exhibited rhythmic contractions that exhibited minimal variance in the time between contractions. Explants treated with 1.5 μM Doxorubicin (right panels) exhibited irregular contractions marked by significantly higher variance in the time between contractions.

FIG. 16. Physcion treatment rescues arrhythmia caused by Doxorubicin. Contractions of atria explants treated with 1.5 μM Doxorubicin+20 μM Physcion (30 sec recordings); x axis=contraction (au); y axis=time (ms). Quantification of contraction rhythmicity of explants was calculated by the COV as described above. Explants treated with Doxorubicin and Physcion regained rhythmic contractions with minimal variance in periods between contractions.

FIG. 17. Physcion does not inhibit Doxorubicin chemotherapeutic effects in vitro. We tested the effects of Physcion and Physcion+Doxorubicin on A549 human epithelial lung cancer cells grown in culture. 5×10⁴ cells were treated with varying doses of Doxorubicin and Physcion for 72 hrs. These studies revealed Physcion alone does not exhibit chemotherapeutic effects. Critically, Physcion concentrations ranging as high as 50 μM did not inhibit the chemotoxic effects of varying doses of Doxorubicin.

FIG. 18. Physcion does not inhibit Doxorubicin chemotherapeutic effects in vivo. We tested the effects of Doxorubicin and Physcion using an A549 xenograft mouse model. Mice with A549 xenograft tumors were treated with vehicle, Doxorubicin, Physcion, or Doxorubicin plus Physcion. Doxorubicin and Physcion treatment was administered by IP injection once a week for two weeks, at a concentration of 4 mg/kg wt for both compounds. Results of these studies showed that Doxorubicin alone effectively mitigates tumor burden, causing a reduction in tumor wt. and volume. Physcion alone did not affect tumor growth. Critically, use of Physcion in conjunction with Doxorubicin did not inhibit the chemotherapeutic effects of Doxorubicin.

### DETAILED DESCRIPTION OF THE DISCLOSURE

Unless defined otherwise, all technical and scientific terms used herein have the same meaning as commonly understood by one of ordinary skill in the art to which this disclosure belongs.

Every numerical range given throughout this specification includes its upper and lower values, all values to the tenth of the lower value, as well as every narrower numerical range that falls within it, as if such narrower numerical ranges were all expressly written herein.

As used in this disclosure including the claims, the singular forms “a”, “an”, and “the” include plural references unless the context clearly dictates otherwise. Thus, for example, references to “the method” includes one or more methods, and/or steps of the type described herein.

The term “therapeutically effective amount” as used herein refers to an amount of an agent sufficient to achieve, in a single or multiple doses, the intended purpose of treatment. Treatment does not have to lead to complete cure, although it may. For example, treatment does not have to lead to complete mitigation of drug-induced cardiac toxicity. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes prophylaxis and treatment of relapse, as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The present disclosure provides compositions and methods for generation of heart pacemaker cells, such as human heart pacemaker cells. The sinoatrial node (SAN) is the primary pacemaker of the heart. The human SAN is poorly understood due to limited primary tissue access and lack of robust in vitro derivation methods. This disclosure provides an efficient method to generate purified human pluripotent stem cell-derived SAN cells (hPSC-SAN). In an embodiment, the method comprises using a reporter for SHOX2 expression, (e.g., dual SHOX2:GFP; MYH6:mCherry knock-in reporter line). The method generates pluripotent stem cell-derived SAN cells displaying molecular and electrophysiological characteristics of SAN cells. These cells can be used for screening of agents that affect SAN cell functioning. For example, therapeutic agents may be screened for side effects on cardiac toxicity, or agents may be identified that can be used as protectants against therapeutic drugs that cause cardiac toxicity. As an example, agents may be identified that can protect against anthracycline-induced cardiac toxicity.

The present method provides an improvement over previous methods. Protze et al. (2017, Nat Biotechnol 35, 56-68) used an NKX2.5-GFP reporter as a negative marker to enrich SAN-like cells, which is far from ideal. Since NKX2.5 is a transcription factor, and not a surface marker, investigators would need to generate such a reporter for any hPSC line they use for differentiation. Also, since it uses negative selection to eliminate non-SAN-like cells (primarily cardiomyocytes) it has limited capacity to expand the desired cell type, rather than eliminate the wrong cell type, which seriously limits capacity to scale. Another method by Schweizer et al. (2017, Stem Cell Res Ther 8, 229) derived pacemaker cells by co-culturing hESC-derived cells with a visceral endoderm-like cell line END-2, which severely limits any application for transplantation or cell therapy purposes. The present method used a dual reporter in pluripotent cells (such as hESC line) to screen for conditions promoting pacemaker differentiation, and to enrich and expand the desired SAN-like population, and established a highly efficient, chemically defined condition to derive functional pacemaker cells. In an embodiment, the dual reporter system SHOX2:eGFP; MYH6:mCherry was used. While the reporter is used for positive output quantification, it is not needed once the protocol is established because it is not a positive selection feature.

The cells used may be human embryonic stem cells or human induced pluripotent stem cells, which are herein together referred to as human pluripotent stem cells (hPSCs). The human pluripotent stem cells may be derived from any suitable cell type, including lung, fibroblasts (e.g. skin fibroblasts), keratinocytes, blood progenitor cells, bone marrow cells, hepatocytes, gastric epithelial cells, pancreatic cells, neural stem cells, B lymphocytes, ES derived somatic cells, and embryonic fibroblasts.

The term “pluripotent stem cell” (also referred to as “PSC”) as used herein refers to a cell having an ability to differentiate into any type of cell of an adult (pluripotency) and also having self-renewal capacity which is an ability to maintain the pluripotency during cell division. “PSCs” include Embryonic Stem Cells (ESCs), which are derived from inner cell mass of blastocysts or morulae, including cells that have been serially passaged as cell lines. Embryonic stem cells, regardless of their source or the particular method used to produce them, can be identified based on their ability to differentiate into cells of all three germ layers, expression of at least Oct4 and alkaline phosphatase, and ability to produce teratomas when transplanted into immunodeficient animals. The term PSCs also includes induced PSCs (iPSCs), which are cells converted from somatic cells by a variety of methods, such as a transient overexpression of a set of transcription factors. A PSC may be a cell of any species with no limitation, and preferably a mammalian cell. It may be a rodent or primate cell. For example, it may be a monkey, mouse or a human pluripotent stem cell. The term “human pluripotent stem cells” or hPSCs includes human embryonic stem cells and human induced PSCs. Human embryonic stem cells may be obtained from established lines of human embryonic stem cells or human embryonic germ cells, such as, for example the human embryonic stem cell lines H1, H7, and H9 (WiCell).

iPSCs can be generated by reprogramming adult cells using lentivirus or plasmids. Any type of human somatic cells (e.g. skin fibroblasts or cells from a biological fluid) can be reprogrammed to iPSC using lentivirus (Maherali et al. Cell Stem Cell 3, 340-345 (2008)) or plasmids (Okita et al. Nat. Methods 8, 409-412 (2011)). For example, human dermal fibroblasts can be infected for a suitable period of time (such as 16 hr) with lentiviruses to express the following: hOct4, hSox4, hKlf4, hNanog, and c-Myc, at specific times. Infected cells can be plated on feeder cells in suitable culture media, and transgenes can be induced. Clones with hESC morphology generally appear after about 3 weeks.

The present disclosure describes a new and efficient strategy to derive and purify SAN-like cells from hPSCs. The ability to generate human SAN-like cells in vitro can facilitate study of genetic and iatrogenic arrhythmias and may allow scaled production and transplantation as biological pacemakers. Our reporter strategy enabled direct visualization and standardized in vitro assays for developmental studies, purification and drug discovery. Using the reporter strategy allowed the development of a method that generated a unique population of SAN-like cells.

The term “sinoatrial node-like cells” or “SAN-like cells” as used herein refers to cardiomyocytes or cardiac cells that express sinoatrial nodal (SAN) cell specific markers TBX18, TBX3, SHOX2 and ISL1 and which are capable of displaying pacemaker activity (e.g., display “funny current”). The SAN cells are the only cells in the heart that can initiate beating, and they alone display a “funny current”, which is a mixed sodium-potassium current that activates upon hyperpolarization at voltages in the diastolic range (normally from −60/−70 mV to −40 mV). The funny current flows through an HCN4-dependent channel. The SAN-like cells express much lower levels of cardiac working muscle markers such as myosins and troponins. Unlike cardiomyocytes, they do not express Cx40.

For culturing of cells, any medium that is routinely used for culturing animal cells can be used, except that no growth factors or serum should be present or are added in the media. Examples of suitable culture media include mTeSR1, Essential 8 medium, BME, F-12, BGJb, MCDB131, CMRL 1066, Glasgow MEM, Improved MEM Zinc Option, IMDM, Medium 199, Eagle MEM, DMEM, Ham, RPMI 1640, and Fischer's media, but other similar media can also be used. Non-growth factor additives, such as antibiotics, B-27 supplement (with or without insulin), amino acids, salts, ascorbic acid and thioglycerol can be added to the media.

Incubation conditions for cell cultures are known in the art. For example, the conditions typically include culturing at a temperature of between about 32-40° C., for example, at least or about 32, 33, 34, 35, 36, 37, 38, 39 or 40° C. The CO2 concentration is generally about 1 to 10%, for example, about 2 to 7%, or about 5% or any range or value between 1 and 10%. The oxygen tension is adjusted to generally to provide normoxic conditions and is preferably about 20%.

The cells, starting with the pluripotent stem cells may be cultured on suitable substrates. For example suitable substrates include Matrigel, collagen IV, fibronectin, laminin, collagen, vitronectin, polylysine, iMatrix-511, iMatrix-521 and the like. These materials are commercially available and routinely used for cell culture.

In an embodiment, the present disclosure provides a method of producing a population of sino-atrial node like cells, comprising or consisting essentially of (a) incubating PSCs with medium comprising or consisting essentially of one or more GSK inhibitors, one or more members of the TGF-β superfamily of proteins, (b) incubating the cells from (a) with a medium comprising or consisting essentially of one or more retinoic acid signaling activators, one or more Wnt inhibitors, one of more ALK inhibitors, and one or more TGF-β superfamily of proteins and/or EGFR inhibitors, and optionally, one or more STAT3 inhibitors, for a period of time to generate SAN progenitor (also referred to as pacemaker progenitor cells that express SHOX2, ISL1 TBX3, TBX5, TBX18 and HCN4; and (c) incubating the SAN progenitor cells in a medium comprising or consisting essentially of one or more GSK inhibitors, one or more EGFR inhibitors, and optionally, one or more HDAC inhibitor for a period of time to generate a population of SAN-like cells that remain enriched for SHOX2, ISL1, TBX3, TBX5, TBX18, and HCN4, but will now also express channels for unique electrophysiological activity. If continued in culture, (such as by 30 days), the cells express CAV3.1, CAV1.3 and Cx30.2 and electrophysiological properties similar to SA node cells. The cells do not express Cx40. The cells may be continued in culture for 30-60 days and beyond that.

Compounds described herein for use for differentiation along the SA node lineage includes pharmaceutically acceptable salts thereof. Other compounds having these functions (i.e., functional analogs) are included within the scope of this disclosure. A “functional analog” as used herein means a compound that has a similar physical, chemical, biochemical, or pharmacological property as compared to another compound. Functional analogs may or may not have similar structures as compared to one another. For example, any functional analog of the particular inhibitors or activators used in the specific embodiments may be used.

Examples of GSK3 inhibitors include CHIR99021, SB2116763, CHIR-98014, and LY2090314.

Examples of Wnt inhibitor include XAV939, IWR-1, IWP-2, and Wnt-C59.

Examples of Activin receptor (ALK) inhibitor include SB431542, LDK378, TAE684, and ASP3026.

Examples of EGFR inhibitors include SU 5402 (Torcis), Tyrphostin AG-490.

Examples of STAT3 (signal transducer and activator of transcription 3) inhibitors include cucurbitacin, Stattic, and Cryptotanshinone.

Examples of HDAC inhibitors include chidamide, Vorinostat, Panobinostat, and Trichostatin A.

Examples of activators of Retinoic acid signaling include retinoic acid (RA), TTNPB, EC23, AM580, Ch55.

Examples of members of the TGF-β superfamily of proteins includes BMP-4 and Activin A.

These inhibitors or activators are generally used at concentrations from 0.01 to 10 μM.

In an embodiment, PSCs may be cultured on a substrate and then upon reaching about 70-80% confluence, incubated with a medium comprising or consisting essentially of a GSK inhibitor, and one or more members of the TGF-β superfamily of proteins for a sufficient period of time (such as 2-3 days) to generate pre-cardiac mesoderm cells. The pre-cardiac mesoderm cells express fetal liver kinase 1 (FLK1) and platelet derived growth factor receptor (PDGFR)-alpha. The pre-cardiac mesoderm cells are incubated in a medium comprising or consisting essentially of a retinoic acid signaling activator, a Wnt inhibitor, an ALK inhibitor, a member of the TGF-β superfamily of proteins and/or an EGFR inhibitor, and optionally, a STAT3 inhibitor, for a sufficient period of time (such as 3 days) to generate pacemaker progenitor cells. The pacemaker progenitor cells express TBX3, TBX5, TBX18, SHOX2, and ISL1. The pacemaker progenitor cells are then incubated in a medium comprising a GSK inhibitor, an EGFR inhibitor (such as 3-9 days), and optionally, a HDAC inhibitor for a sufficient period of time to generate SA-node like cells. The HDAC inhibitor may be added at the same time as the GSK inhibitor and the EGFR inhibitor or maybe added subsequent to an initial culture period with GSK inhibitor and the EGFR inhibitor (such as initial period of 3-6 days with GSK inhibitor and EGFR inhibitor, and another 1-3 days with GSK inhibitor, EGFR inhibitor and HDAC inhibitor). The SA-node like cells express SHOX2, TBX5, TBX3, ISL1 and TBX18, as well as CAV3.1, CAV1.3 and Cx30.2, and HCN4, and display the funny current based on electrophysiological analysis The SA-node like cells can be continued in culture in a serum-free, growth factor free medium for an extended period of time (such as at least 3 months).

In embodiments, the various factors referred to for the steps may be the only differentiation/growth factors present in the culture medium. For example, PSCs may be incubated in a medium in which the only differentiation factors are a GSK inhibitor, and one or more members of the TGF-β superfamily of proteins to generate pre-cardiac mesodermal cells, the pre-cardiac mesodermal cells may be incubated in a medium in which the only differentiation factors are retinoic acid signaling activator, Wnt inhibitor, ALK inhibitor, a member of the TGF-β superfamily of proteins and/or an EGFR inhibitor, and optionally, STAT3 to generate pacemaker progenitor cells, and the pacemaker progenitor cells may be incubated in a medium in which the only differentiation factors are GSK inhibitor, an EGFR inhibitor, and optionally, a HDAC inhibitor to generate SAN-like cells. Non-growth/differentiation factor additives, such as antibiotics, B-27 supplement (with or without insulin), amino acids, salts, ascorbic acid and thioglycerol, may be present.

In an embodiment, PSCs may be cultured on a substrate and then upon reaching about 70-80% confluence, incubated with a medium comprising or consisting essentially of a CHIR99021, BMP4 and/or Activin A for a sufficient period of time (such as 3 days) to generate pre-cardiac mesoderm cells. The pre-cardiac mesoderm cells are incubated in a medium comprising or consisting essentially of cucurbitacin, retinoic acid, XAV939, SB431542, MP4 and/or μM SU5402 for a sufficient period of time (such as 3-6 days) to generate pacemaker fate cells. The pacemaker fate cells are then incubated in a medium comprising or consisting essentially of a CHIR99021, Tyrphostin AG 490 and/or 1-10 μM chidamide for a sufficient period of time (such as 1-3 days) to generate SA-node like cells. The SAN-like cells exhibit SAN-like electrophysiological characteristics, such as ‘funny channel’ by 30 days (total time in culture, starting from PSCs). The SA-node like cells can be continued in culture for an extended period of time (such as at least 3 months). It was observed that while it was preferred for some cell lines to include both BMP-4 and Activin A in the medium for generating pre-cardiac mesoderm cells, in other cell lines only BMP-4 or Activin A was sufficient. Thus, these components may be optimized for a particular cell line.

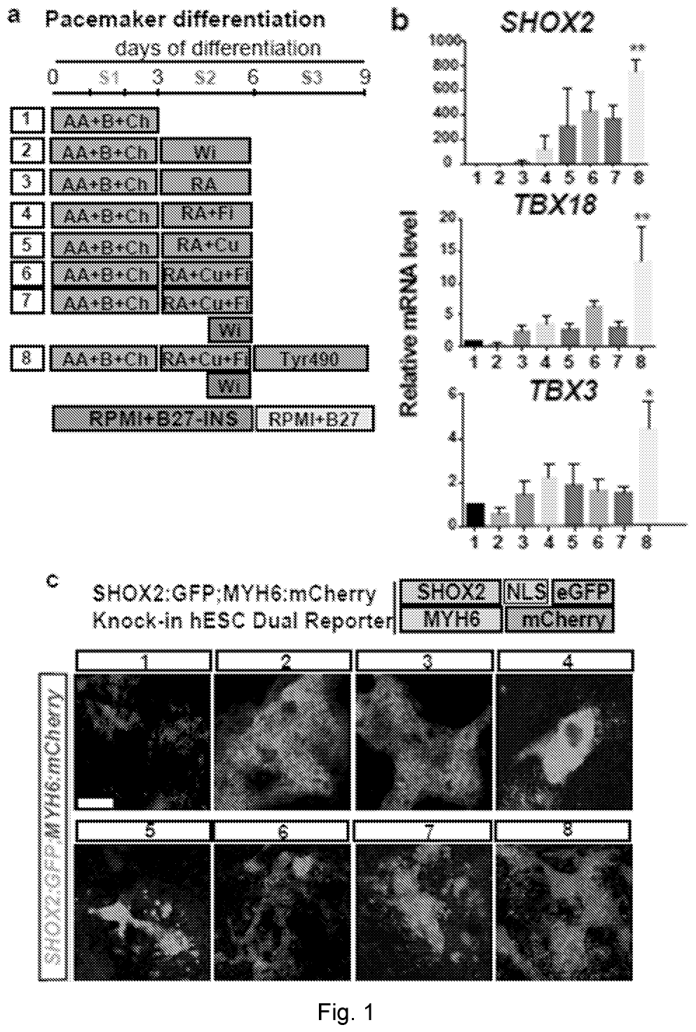

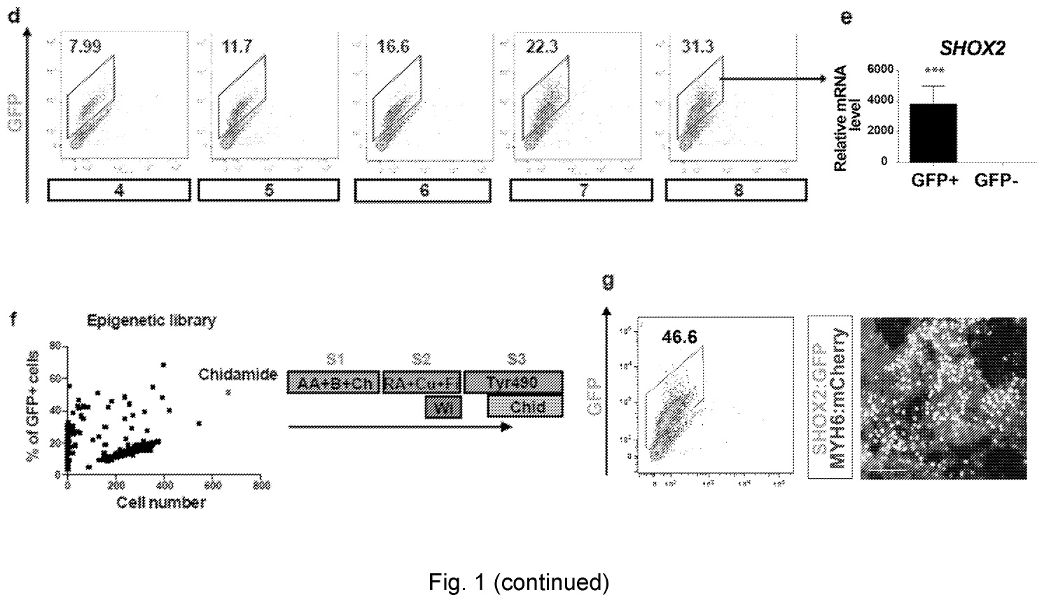

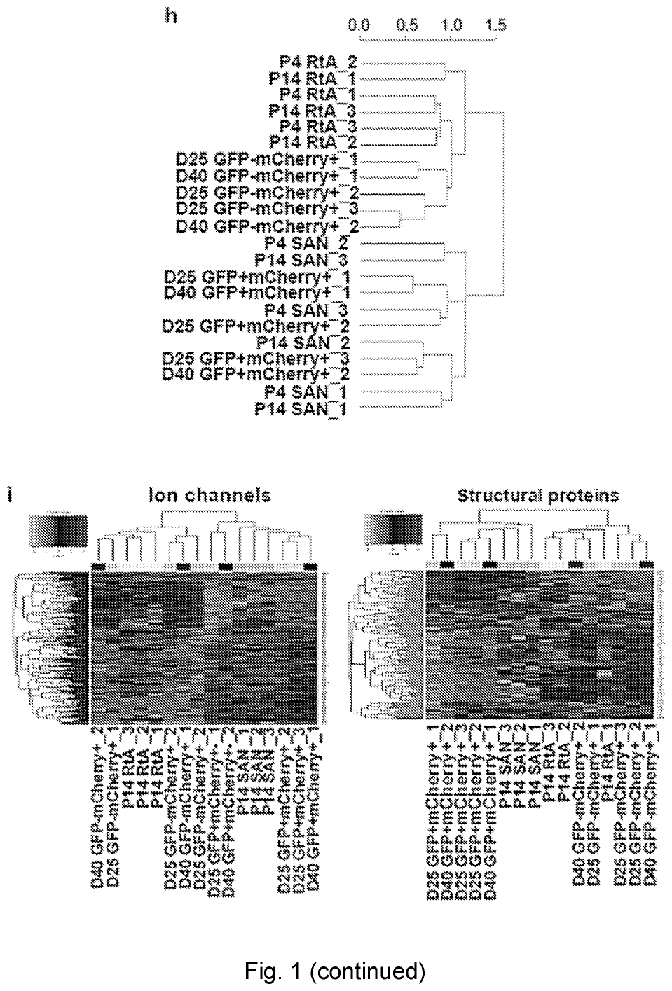

In an embodiment, PSCs may be cultured on a substrate and then upon reaching about 80% confluence (in about 72 hours), incubated with a medium comprising or consisting essentially of a 0.5-3 μM CHIR99021, 5-100 ng/mL BMP4 and/or 5-50 ng/mL Activin A in RPMI (Cellgro) supplemented with B27 minus insulin, 2 mM GlutaMAX, 1× NEAA and/or 1× Pen/Strep for 3 days (RB27-INS) to generate pre-cardiac mesoderm cells. The pre-cardiac mesoderm cells are incubated in a medium comprising or consisting essentially of 0-5 M cucurbitacin, 0.1-10 μM retinoic acid, 1-10 μM XAV939, 1-10 μM SB431542, 5-100 ng/mL BMP4 and/or 0.1-10 μM SU5402 in RB27-INS for the next 3-6 days to generate pacemaker fate cells. The pacemaker fate cells are then incubated in a medium comprising or consisting essentially of a 0.5-3 μM CHIR99021, 1-10 μM Tyrphostin AG 490 and optionally, 1-10 μM chidamide in RB27-INS from day 7 to day 9 to generate SA-node like cells. The SA-node like cells can be continued in culture in RPMI+B27 medium. for an extended period of time (such as at least 3 months). Variations of the protocol are shown in FIG. 1a, where Ch is CHIR99201, B is BMP-4, AA is Activin A, Cu is cucurbitacin, RA is retinoic acid, Wi is XAV939, Fi is SU5402 and Tyr490 is tyrphostin AG-490.

In an embodiment, of the many potential applications, we used the hPSC-SAN platform for in vitro prediction of pacemaker cell toxicity following anthracycline treatment, since supraventricular arrhythmias and/or atrial fibrillation can be early manifestations of the toxicity. One striking finding from the present results was a significant sensitivity to DOXO treatment in cell lines harboring genetic variation in the CBR3 locus. The platform also identified new loci that may potentially be associated with increased arrhythmia susceptibility in patients receiving anthracyclines. This platform can be exploited as a high throughput in vitro safety test for future potential drugs that might affect SAN-like cells. Finally, the ability of physcion to modulate doxorubicin-related toxicity on SAN-like cells represents proof-of-concept for the use of human SAN-like cells for the identification of compounds that can counteract arrhythmogenic drugs.

In an aspect, this disclosure provides a method of selectively producing and identifying SAN-like cells comprising providing a population of human pluripotent stem cells and introducing into the cells, a reporter for SHOX2 expression, and based on the expression of SHOX2, identifying and isolating hPSC-SAN cells. In an embodiment, the method comprises i)) providing a population of human pluripotent stem cells, ii) introducing into the cells, a reporter for SHOX2 expression, iii) providing the cells from ii) with a GSK inhibitor, and one or more members of TGF-β superfamily of proteins, iv) providing cells from iii) with retinoic acid, Wnt inhibitor, ALK inhibitor, a TGF-β superfamily of proteins and/or EGFR inhibitor, and optionally, a STAT3 inhibitor, v) providing the cells from iv) with GSK inhibitor and EGFR inhibitor, and optionally, additionally providing to the cells with an HDAC inhibitor, and vi) sorting for cells that are positive for the reporter for SHOX2. The cells may be used for implantation or for screening (such as in a high throughput screening) to identify agents that can affect the functioning of SAN cells.

Introduction of the reporter, such as SHOX2 permits the refinement of the technique and also allows for validation. However, the introduction of the reporter is not required for the generation of SAN-like cells from PSCs. Thus the steps of the method may be carried out without introducing the reporter for SHOX2 and without screening for the presence of SHOX2.

In an aspect, this disclosure provides a population of cells derived from human pluripotent stem cells which exhibit SAN like characteristics. If the SAN-like cells are derived from hPSCs, they may be referred to as hPSC-SAN cells or hPSC-SAN-like cells. In embodiments, the SAN-like cells, such as hPSC-SAN cells, may be at least 50% pure in the population. In embodiments, the population may be at least 60%, at least 70%, at least 80%, at least 90% or at least 95% or 99% pure for SAN-like cells, such as hPSC-SAN cells. In an embodiment, this disclosure provides a population of cells produced by the methods described herein.

In an aspect, this disclosure provides a method for identifying drugs that induce cardiac toxicity by exposing the candidate agent to a population of hPSC-SAN cells generated as described in the present disclosure and evaluating for effects on the functioning of the cells. The functioning may be evaluated in terms of chemical, biological, biochemical or electrophysiological characteristics.

In an aspect, this disclosure provides a method for identifying agents that can protect against drug induced cardiac toxicity by exposing the particular drug or therapeutic agent to a population of hPSC-SAN cells in the presence or absence of candidate protecting agents and evaluating for effects on the functioning of the SAN-like cells, and identifying agents that minimize or eliminate adverse effects of the therapeutic agent. The functioning may be evaluated in terms of chemical, biological, physical, biochemical or electrophysiological characteristics.

In an aspect, this disclosure provides a method of reducing cardiac toxicity side effects of a therapeutic agent comprising administering to an individual in need of treatment the therapeutic agent and a cardiac toxicity protective agent, which exhibits protection action on SAN-like cells. An example of a cardiac toxicity protective agent is physcion.

In an embodiment, this disclosure provides a method of treatment of cancer by chemotherapeutic agents comprising administering a chemotherapeutic agent in conjunction with a cardiac toxicity protective agent, wherein the cardiac toxicity protective agent reduces cardiac toxicity side effect of the chemotherapeutic agent, but does not adversely affect its cell growth inhibiting efficacy. In an embodiment, the chemotherapeutic agent may be an anthracycline (such as doxorubicin) and the cardiac toxicity protective agent may be physcion.

In an embodiment, this disclosure provides compositions and methods for the treatment of cardiac toxicity, the compositions comprising a therapeutically effective dose of physcion or derivatives thereof. The compositions may further comprise pharmaceutically acceptable carriers, additives, excipients, stabilizers, enhancers, adjuvants and the like. A therapeutically effective amount of physcion may be determined by a person skilled in the art given the benefit of the present disclosure and known principles of clinical therapeutics. In embodiments, compositions comprising physcion may be administered in conjunction with the therapeutic agent whose cardiac toxicity is desired to be muted. The two may be administered together or separately, by the same route or different routes, and over the same period of time or different periods of time.

The structure of physcion is as follows.

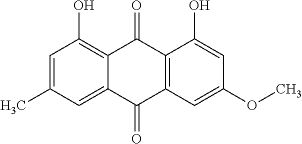

Derivatives of physcion include glycosylation at either of the alcohols, general functionalization on the alcohols with protecting groups (e.g., trimethyl silyl groups or acetyl groups), and dimerization of physcion (either through one of the carbonyls of the anthraquinone or through substitution on one of the flanking rings). Physcion derivatives may be produced by derivatizing physcion at one or more of its four main substituents (the methoxy group, two alcohol groups, and methyl group) providing a structure as follows.

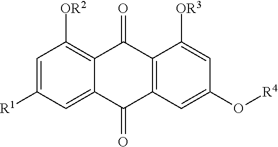

where R¹ and R⁴ are chosen from substituted and unsubstituted alkyl groups and substituted and unsubstituted alkoxy groups, and R² and R³ are chosen from hydrogen, substituted and unsubstituted alkyl groups, substituted and unsubstituted alkylcarbonyl groups, substituted and unsubstituted alkylsulfonyl groups, and substituted and unsubstituted alkylphosphonyl groups.

Physcion and/or derivatives thereof may be provided as pharmaceutical compositions comprising pharmaceutically suitable carriers. Suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG). Examples of suitable pharmaceutical preparation components can be found in Remington: The Science and Practice of Pharmacy 20th edition (2000). The pharmaceutical compositions may comprise other therapeutic agents.

The subject to be treated may be an individual who has one or the more of the following SNPs: a) rs1056892 in CBR3, b) rs2229774 in RARG, c) rs885004 in SLC28A3, d) rs9559211 in LIG4, e) rs7314566 in ANO2, f) rs17267852 in NRXN1.

In an aspect, the present invention provides a method for treating a subject suffering from a defect related to SA-node malfunction. The method comprises culturing pluripotent stem cells, differentiating the pluripotent stem cells in vitro into SAN-like cells, and implanting the cells into the SA-node area within the heart of the individual.

Cells may be implanted into an appropriate site in a recipient. The amount of cells used in implantation depends on a number of various factors including the patient's condition and response to the therapy, and can be determined by one skilled in the art. The number of cells for transplantation may be used from 1 million to 20 million and all integer values therebetween. In embodiments, the number of cells used may be 1, 2, 5, 7.5, 10, 12.5, 15, 17.5 and 20 million cells. In an example, the differentiated SAN-like cells may be implanted as dispersed cells or formed into clusters that may be infused into the appropriate cardiac site via a suitable method, such as catheterization. Alternatively, cells may be provided in biocompatible degradable polymeric supports, porous non-degradable devices or encapsulated to protect from host immune response. Support materials suitable for use for implantation of cells of the present disclosure include tissue templates, conduits, barriers, and reservoirs, such as those used for tissue repair. For example, synthetic and natural materials in the form of foams, sponges, gels, hydrogels, textiles, and nonwoven structures may be used.

Some examples in the following paragraphs provide non-limiting embodiments of the present disclosure.

Example 1) A method to generate cells with sinoatrial node (SAN) characteristics from human pluripotent stem cells comprising: a) providing a population of human pluripotent stem cells; b) transfecting the cells with a reporter for SHOX2 expression;

c) providing retinoic acid, an EGF inhibitor, an FGF inhibitor, and a STAT3 inhibitor; d) providing an HDAC inhibitor; and e) sorting for cells positive for the reporter for SHOX2.

Example 2) The method of Example 1, wherein the initial population of cells are embryonic pluripotent cells.

Example 3) The method of Example 1 wherein the initial population of cells are induced pluripotent cells.

Example 4) The method of Example 1, wherein 5 μM EGF inhibitor is used, and said inhibitor is Tyrphostin AG490.

Example 5) The method of Example 1 wherein 0.1 μM STAT3 inhibitor is used, and said inhibitor is cucurbitacin.

Example 6) The method of Example 1 wherein 5 μM of FGFR inhibitor is used, and said inhibitor is SU5402.

Example 7) The method of Example 1 wherein 1 μM RA is used.

Example 8) The method of Example 1, where cells are incubated with X for about 7 days, after which the HDAC inhibitor is administered.

Example 9) The method of Example 1 wherein the HDAC inhibitor is chidamide.

Example 10) The method of Example 1, where the sorting in Example if occurs around 45 days after step 1d.

Example 11) A plasmid encoding SHOX2:GFP

Example 12) A method to test drugs for cardiac side effects by testing them on the hPSC-SAN cells.

Example 13) A method to screen for drug leads using the hPSC-SAN cells, including drug leads that prevent cardiac side effects of other drugs.

Example 14) A method to prevent anthracycline-induced cardiotoxity by co-administering physcion.

Example 15) Method of Example 14, wherein the subject has any of the following SNPs: a) rs1056892 in CBR3, b) rs2229774 in RARG, c) rs885004 in SLC28A3, d) rs9559211 in LIG4, e) rs7314566 in ANO2, and f) rs17267852 in NRXN1.

The following examples are provided for illustrative purposes and not intended to be limiting.

### Example 1

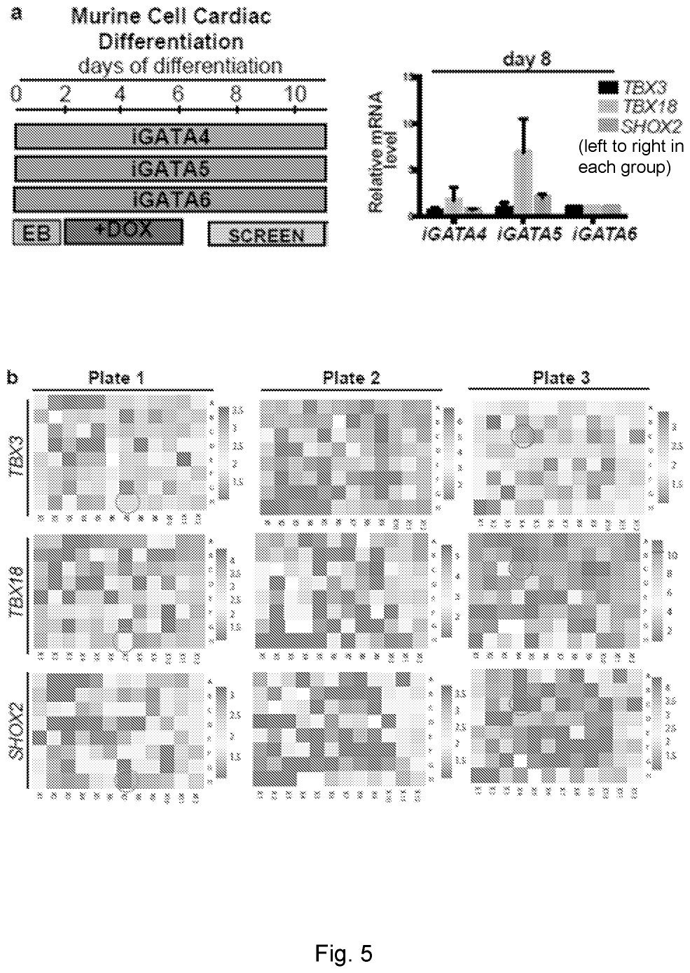

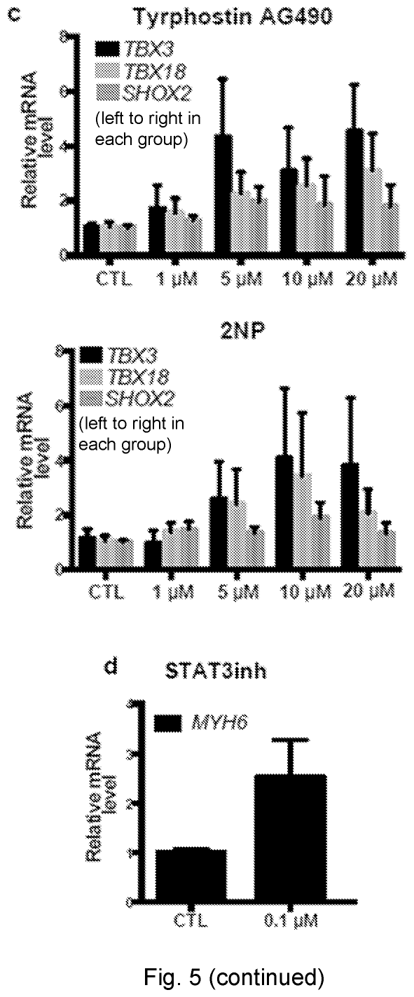

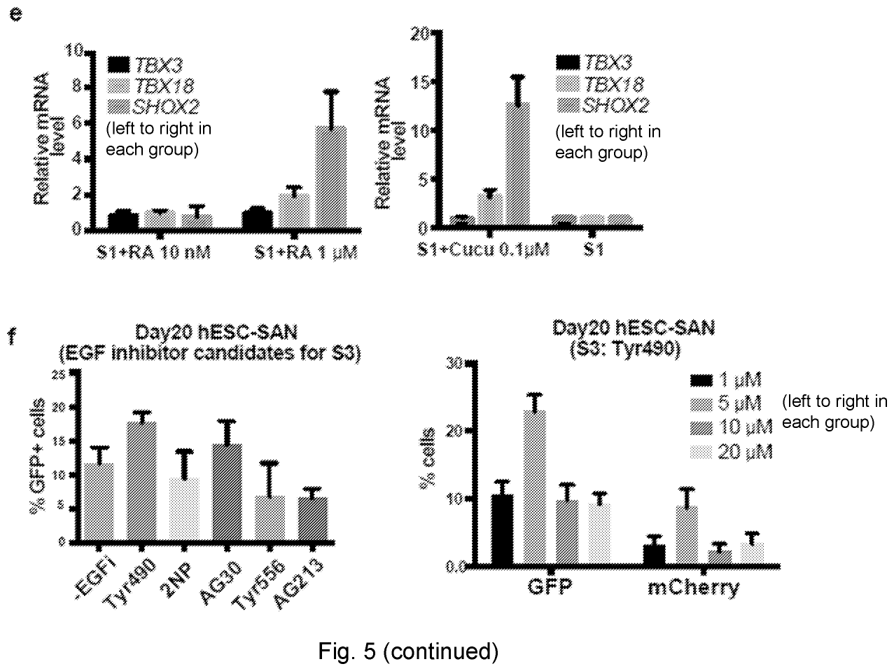

We developed a protocol that maximizes the generation of pluripotent stem cell-derived SAN pacemaker lineages (PSC-SAN). Based on previous reports (Hoogaars et al., Genes Dev 21, 1098-1112 (2007); Ionta et al., Stem Cell Reports 4, 129-142 (2015); McNally et al., Circ Res 104, 285-287 (2009), SAN progenitors are expected to be enriched for expression of key pacemaker transcription factors TBX3, TBX18, and SHOX2 (TTS). Recently, murine embryonic stem cell (ESC) lines that were engineered for conditional expression of GATA4, GATA5 or GATA6, to efficiently generate diverse cardiac cell populations including, nodal, atrial and ventricular cells⁴. Among these cell lines, GATA5 induction resulted in the highest expression levels of TTS (FIG. 5a). We therefore applied GATA5-induced progenitors to an unbiased qRT-PCR-based chemical screen using annotated pathway modulating compounds to identify modulators that further enhance the expression of TTS and specification toward pacemaker lineage. Two compounds caused a minimum of 2-fold increase in transcript levels for at least two of the transcription factors (FIG. 5b). Both were EGF pathway inhibitors (FIG. 5c), suggesting an important role for the EGF pathway in nodal identity. In addition to transcription factors, we also focused on the chemicals that increased the expression of cardiac lineage structural proteins. The screen identified cucurbitacin, a STAT3 inhibitor, that enhances the expression levels of nodal and atrial structural marker MYH6 (FIG. 5d).

These results were next translated into a human embryonic stem cell (hESC) differentiation protocol, including two additional pathway modulators. Retinoic acid (RA) was included to shift regional identity of cardiac progenitors toward a more caudal phenotype, since pacemaker cells are thought to arise from a more caudal population of cardiac progenitors. In addition, an FGF inhibitor was used to augment the expression of ISL1, a known regulator of the nodal program in early mouse development. Indeed, the full combination (condition 8) of 5 μM EGF inhibitor (Tyrphostin AG490), 0.1 μM STAT3 inhibitor (cucurbitacin), 5 μM FGFR inhibitor (SU5402), and 1 μM RA significantly increased human TTS expression levels (FIG. 1a, b, and FIG. 5e, f).

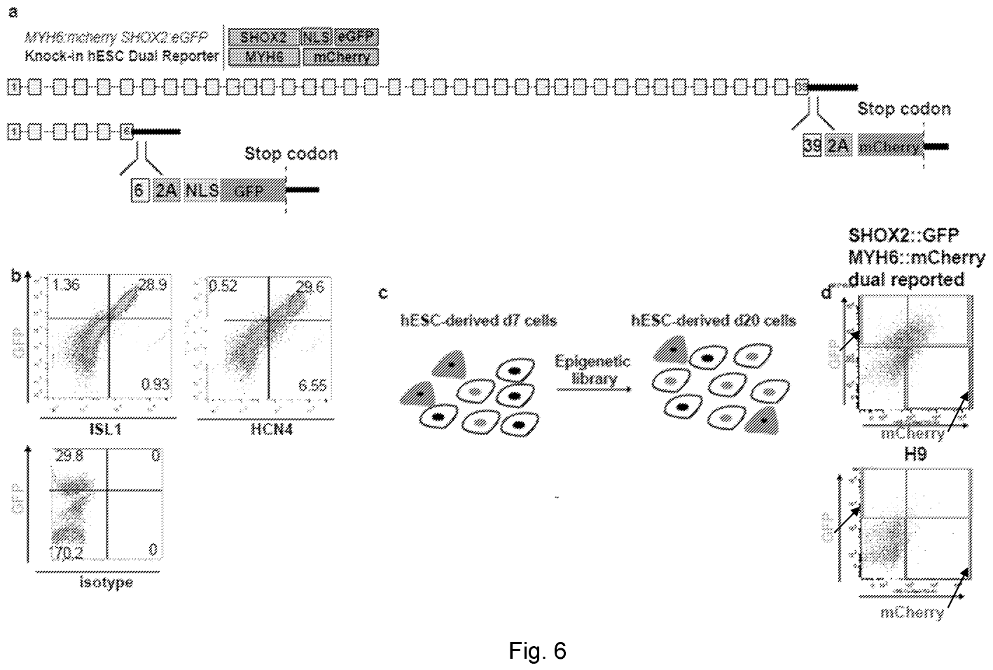

To quantify and purify SAN-like cells using this directed differentiation protocol, a dual knock-in SHOX2:GFP; MYH6:mCherry reporter line was created using CRISPR/Cas-based gene-targeting techniques (FIG. 6b). Immunofluorescence and FACS analyses confirmed the success of the protocol for generating putative hPSC-SAN cells, as condition 8 maximizes generation of SHOX2:GFP⁺ cells (FIG. 1c, d). The sorted GFP⁺ cells were enriched approximately 4000-fold for SHOX2 transcript levels compared to GFP⁻ cells (FIG. 1e), and most of the GFP⁺ cells co-express ISL1 and HCN4, proteins closely associated with the development and function of SAN cells (FIG. 6b). To further enhance production of GFP⁺ cells, we considered that SAN development may also be controlled epigenetically and performed an unbiased screen using a library of known epigenetic modulatory compounds. Cells were differentiated using condition 8 with compounds added at day 7 (FIG. 1f and FIG. 6c). After 14 days of differentiation, treatment with the HDAC inhibitor chidamide led to a significant increase in the percentage of GFP⁺ cells (˜50%) without reducing the total cell number, demonstrated by both flow cytometry and immunostaining (FIG. 1g).

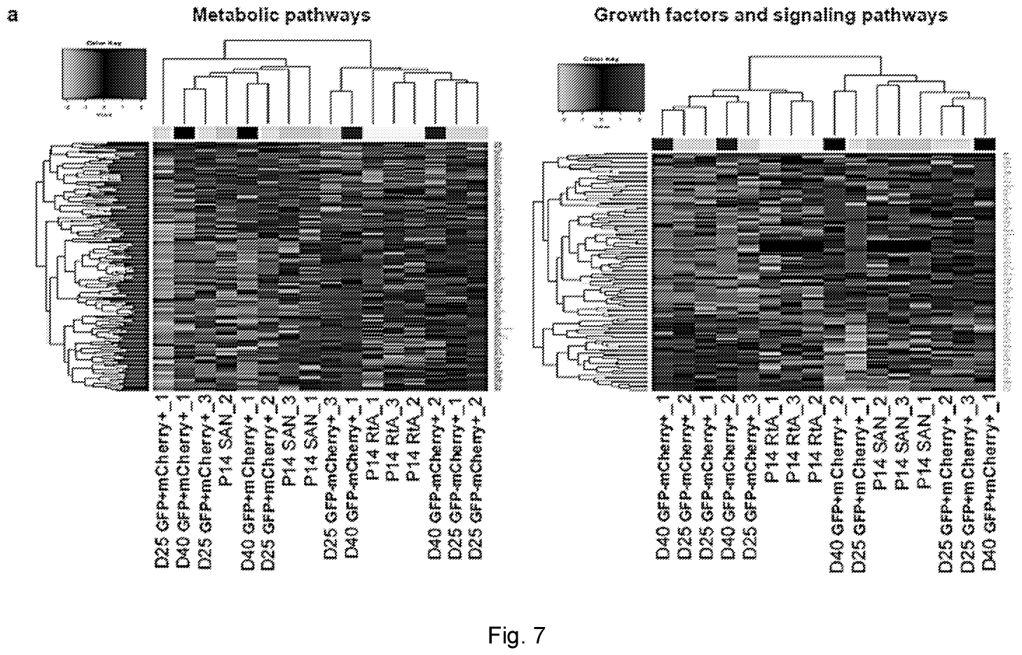

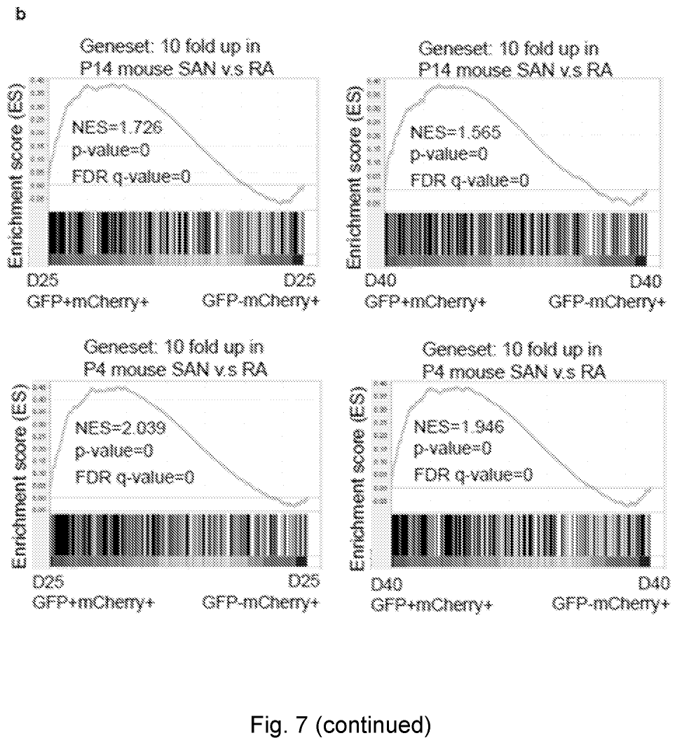

To better validate the nodal identity of the SHOX2:GFP⁺ cells, the GFP⁺ (regardless of mCherry expression) and GFP⁻mCherry⁺ populations were sorted at Day 25 or Day 40 of differentiation and analyzed by RNA-seq. The data was compared by hierarchical clustering with RNA-seq databases generated using HCN4:GFP⁺ cells from dissected murine SAN tissue and GFP⁻ cells from adjacent right atrial tissue (GSE65658). Whether the clustering analysis used whole transcriptomic data (FIG. 1h) or selected datasets for genes encoding ion channels, structural proteins (FIG. 1i), metabolic proteins, or growth factors (FIG. 7a) the GFP⁺ and GFP⁻mCherry⁺ cells clustered with SAN and atrial cardiomyocytes, respectively. The phenotypes were further confirmed by gene set enrichment analyses: the geneset representing genes with levels enhanced 10-fold in SAN vs atrial cells were highly enriched in GFP⁺ cells while those that have levels enhanced 10-fold in atrial vs SAN cells were highly enriched in GFP⁻mCherry⁺ cells (FIG. 7b).

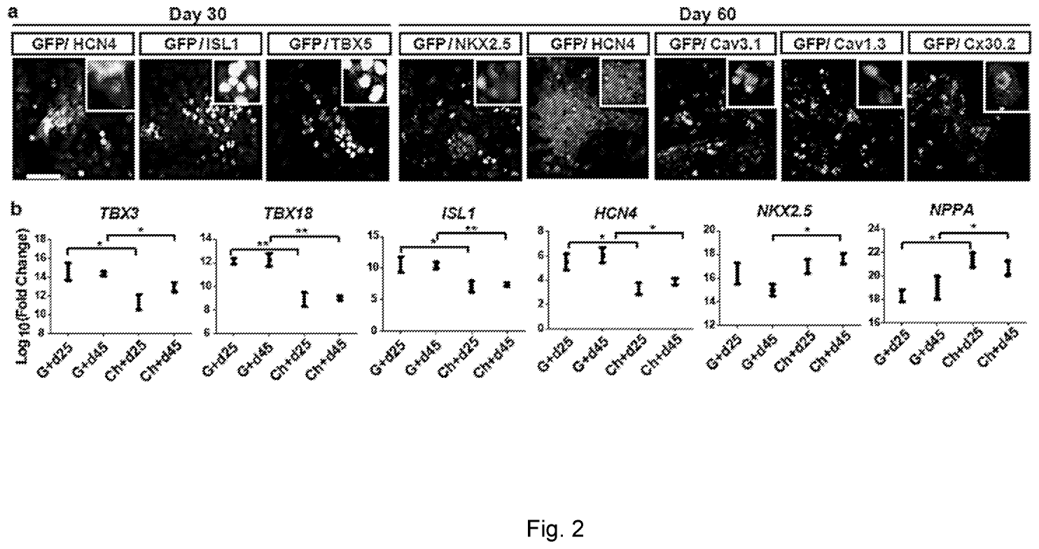

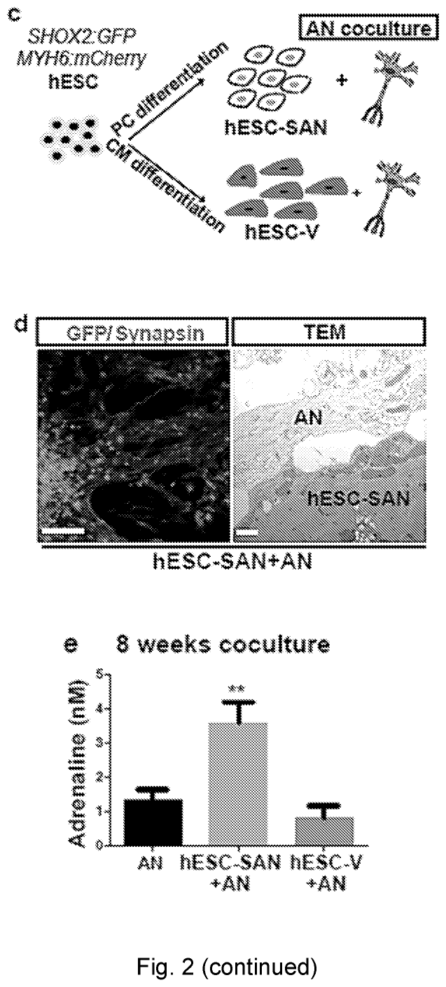

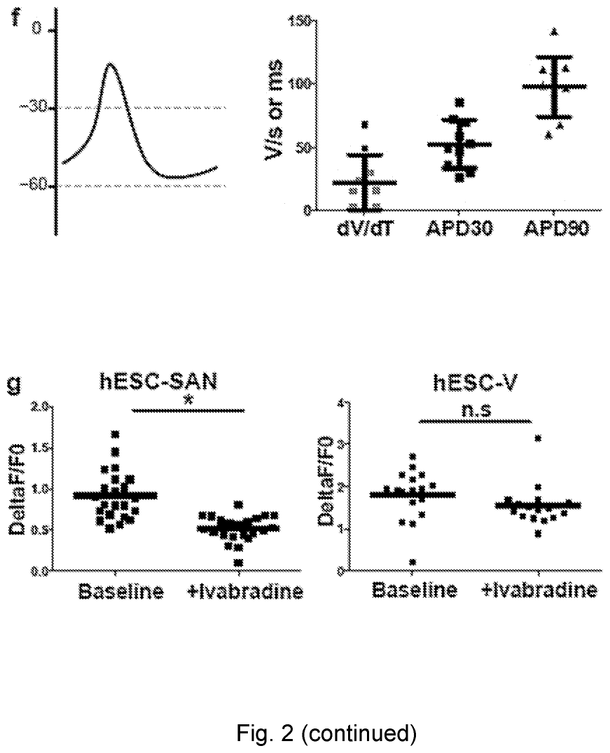

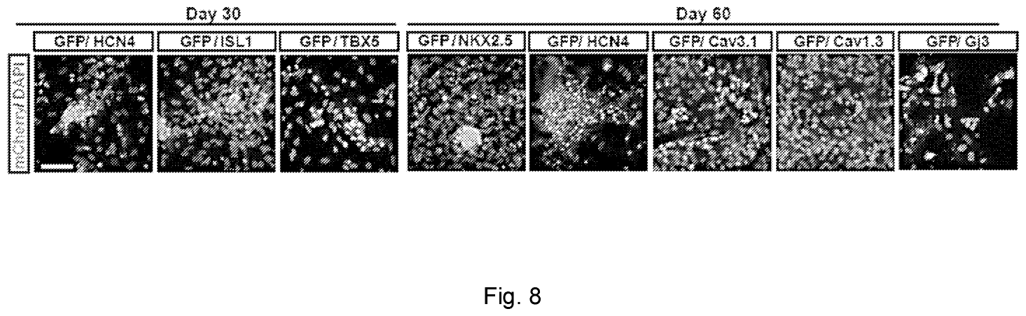

We maintained differentiated cells for up to 60 days and evaluated gene expression patterns. At day 30, most SHOX2:GFP⁺ cells stained positive by immuno-fluorescence for ISL1, TBX18, TBX5 and HCN4 (FIG. 2a, and FIG. 8 left panel). By day 60, there was increased expression levels for channels related to pacemaker activity, including CAV3.1, CAV1.3, CX30.2 (FIG. 2a, and FIG. 8 right panel). Transcript levels of pacemaker markers TBX3, TBX18, ISL1, and HCN4 increased over time, while decreasing for the atrial cardiomyocyte marker Nppa in SHOX2:GFP⁺; MYH6:mCherry⁺ cells compared to SHOX2:GFP; MYH6:mCherry⁺ cells (FIG. 2b). There was no significant difference in NKX2.5 expression levels comparing these two populations until day 45. This is in agreement with studies showing that NKX2.5 and SHOX2 are co-expressed during the early stages of differentiating embryoid bodies.

A major function of the SAN is to regulate heart rhythm under the control of autonomic neurons (AN). ESC-derived cardiomyocytes show poor connectivity with ESC-derived AN¹². We probed the functionality of in vitro-derived hPSC-SAN cells by assessing connectivity with AN (FIG. 2c, d). For connectivity studies, an optogenetic reporter line was used to facilitate light-induced control of neural activity. AN were derived from a hESC line expressing enhanced yellow fluorescent protein (eYFP)-tagged channelrhodopsin-2 (ChR2) under control of the human synapsin promoter. Co-culture of day 25 hPSC-SAN cells with these AN resulted in physical connection between SAN-like cells and AN, as shown by increased expression of synapsin, and junctional morphology indicated by electron microscopy (FIG. 2d). Furthermore, the co-culture platform resulted in increased maturation of AN as shown by increased adrenaline levels in supernatant compared to AN co-cultured with hESC-derived ventricular-like cardiomyocytes (FIG. 2e). Light-activated contractions in SAN-like cells further validated the connectivity.

One key functional property of a nodal cell is its characteristic action potential with fast spontaneous firing rates and slow maximum upstroke velocities (<30 V/s). SHOX2:GFP⁺ cells generated from the differentiation protocol at day 30 were sorted and patch-clamping experiments showed 8 out of 10 demonstrated the characteristic electrophysiologic phenotype of nodal cells (FIG. 2f). Functional hPSC-SAN cells should provide a platform to test rhythm-modifying drugs. Ivabradine, a specific HCN4 channel modulator, has been shown to slow SAN activity. Directed differentiation was used to generate hESC-SAN or hESC-derived ventricular myocytes (hESC-V) and a Ca²⁺-flux assay was used to record the baseline calcium activity of each cell type. The cells were then treated with 1 μM ivabradine and recordings taken after 5 min. While no significant difference was observed in cardiomyocyte cultures, a significant reduction was observed in contractions and calcium activity in the hESC-SAN cells (FIG. 2g). Therefore, based on multiple criteria, including gene expression profiles, AN connectivity, electrophysiological characteristics, and drug sensitivity, the hESC-SAN cells recapitulate expected features of human pacemaker cells.

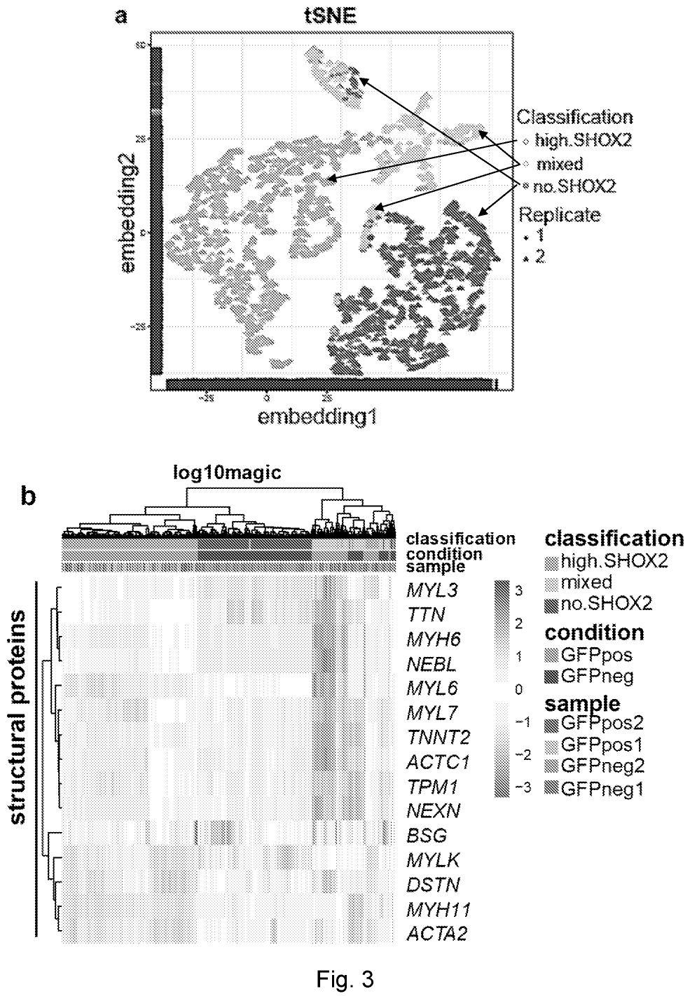

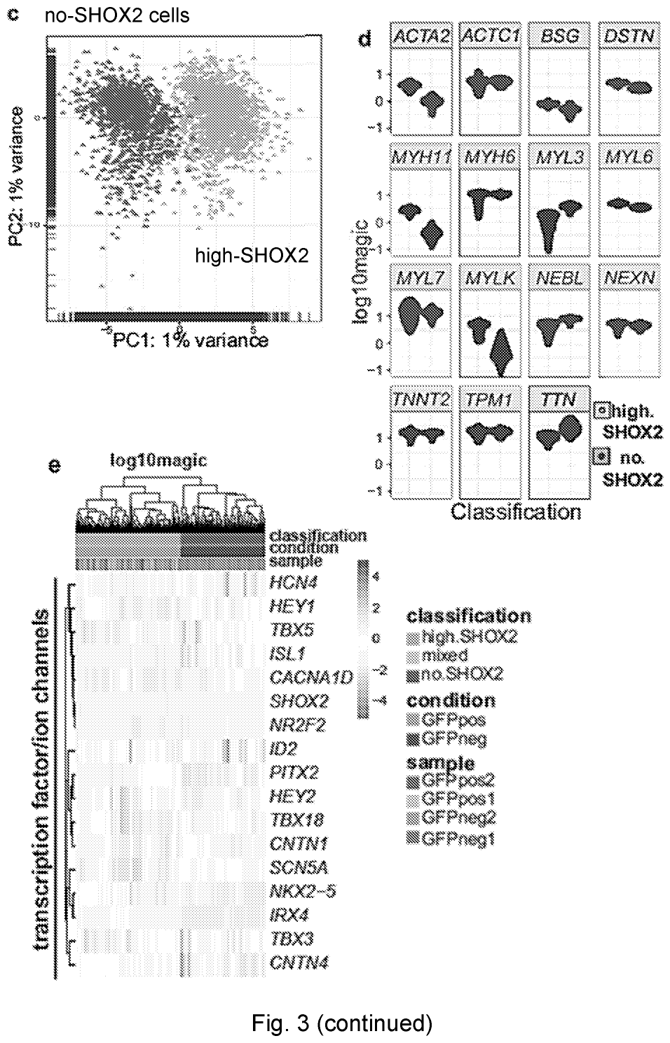

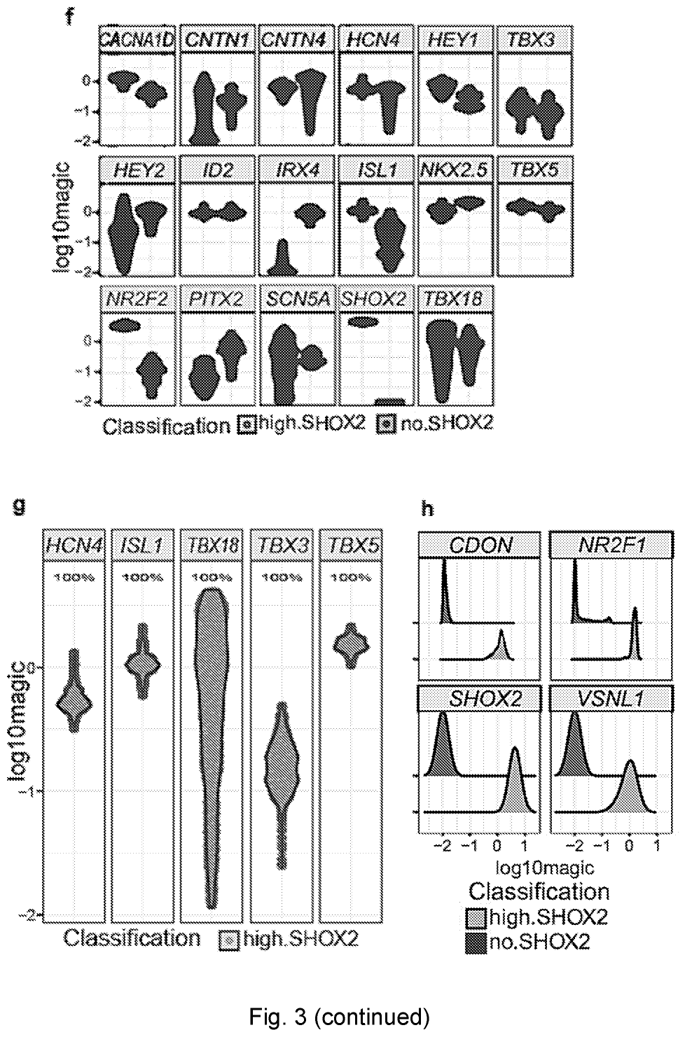

The hPSC-SAN cells were analyzed in a single-cell RNAseq experiment to explore heterogeneity of the cell population and identify lineage-restricted transcriptome features. For this purpose SHOX2:GFP⁺ cells in the hPSC-SAN population were sorted from the pacemaker differentiation culture while MYH6:mCherry⁺ cells were sorted following cardiomyocyte directed differentiation to generate a ventricular-like population (hESC-V). After pooled sequencing, tSNE plots (FIG. 3a) clustered cells with similar gene expression patterns. Cluster analysis using global gene expression values classified the cells into three groups including high-SHOX2, mixed-SHOX2 and no-SHOX2. Since cardiac markers are not highly expressed in the mixed-SHOX2 population, these cells might represent non-cardiac SHOX2⁺ cells (FIG. 3b). We therefore focused on high-SHOX2 and no-SHOX2 cells for further analysis. Principal component analysis (PCA, FIG. 3c) confirmed that high-SHOX2 and no-SHOX2 cells are two distinct populations (FIG. 3c).

High-SHOX2 cells were significantly enriched in transcripts encoding structural proteins TPM1, TNNT2, MYL7, MYH6, MYL6, ACTA2, DSTN, ACTC1, NEXN, NEBL, MYH11 and MYLK (FIG. 3d). The role in SAN physiology for some of these proteins including MYLK, MYH11 and ACTA2 is unknown, although they were also reported to be present in the murine SA node¹⁰. Notable structural protein genes expressed in the no-SHOX2 cell population encode TTN, TPM1, TNNT2, MYL7, MYH6 and ACTC1. The presence of IRX4 transcripts in the no-SHOX2 cells (and absence in the high-SHOX2 cells) confirms the ventricular identity of the former (FIG. 3d). The high-SHOX2 cell population was relatively homogenous in expression of transcripts for the key ion channel HCN4 and transcriptional regulator ISL1, while no-SHOX2 cells showed heterogeneity in the expression levels of these two genes (FIG. 3e). PITX2c, a gene responsible for suppression of the SA program in atrial cells, was expressed in the no-SHOX2 and not in the high-SHOX2 cells (FIG. 3f). The high-SHOX2 cell population had lower but detectable NKX2.5 expression levels, and was also associated with relatively higher levels of HEY1 and ID2 transcripts, compatible with an SA nodal identity rather than an AV node. Consistent with previous studies, CACNA1D and SCN5A channels, which are necessary for the peacemaking activity of the nodal cells, were relatively enriched in high-SHOX2 cells. Among contacting isoforms, CNTN4 transcripts were relatively more abundant in the high-SHOX2 cell population, compared to dispersed expression levels of CNTN1.

The high-SHOX2 population showed relatively homogenous expression of HCN4, ISL1, TBX5 and TBX3 genes compared to TBX18 (FIG. 3g). Previous murine developmental studies suggested that the TBX18 expression pattern might determine the identity of different parts of the SA node, including head or tail. The single cell sequencing data indicates that the in vitro generated hPSC-SAN cells recapitulate a spectrum of TBX18 expression patterns, and therefore may represent multiple segments of the SA node. Another key transcriptional regulator identified in high-SHOX2 cells was NR2F2 (COUPTFII), known to be important for the development of cardiac cells with caudal identity (FIG. 3h).

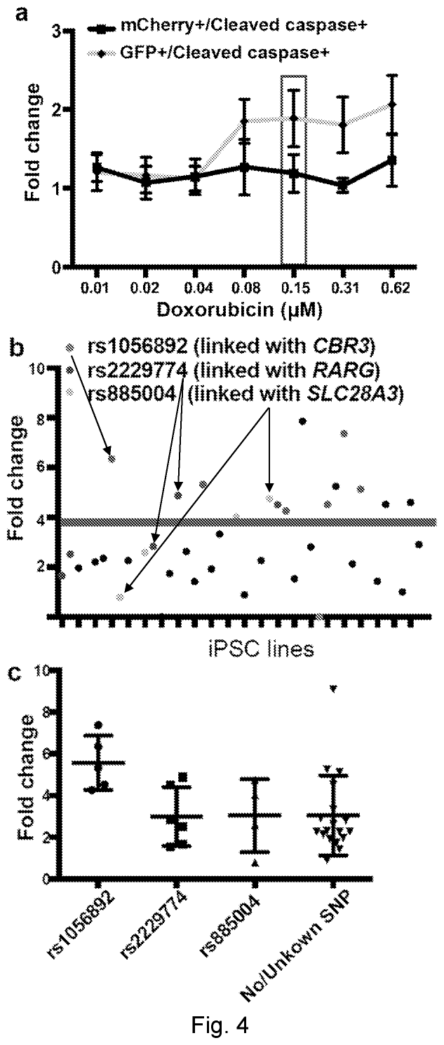

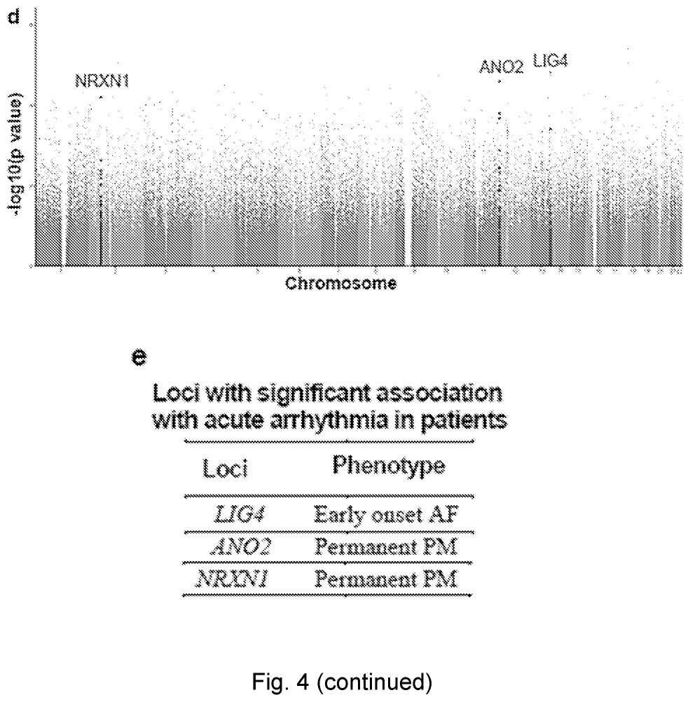

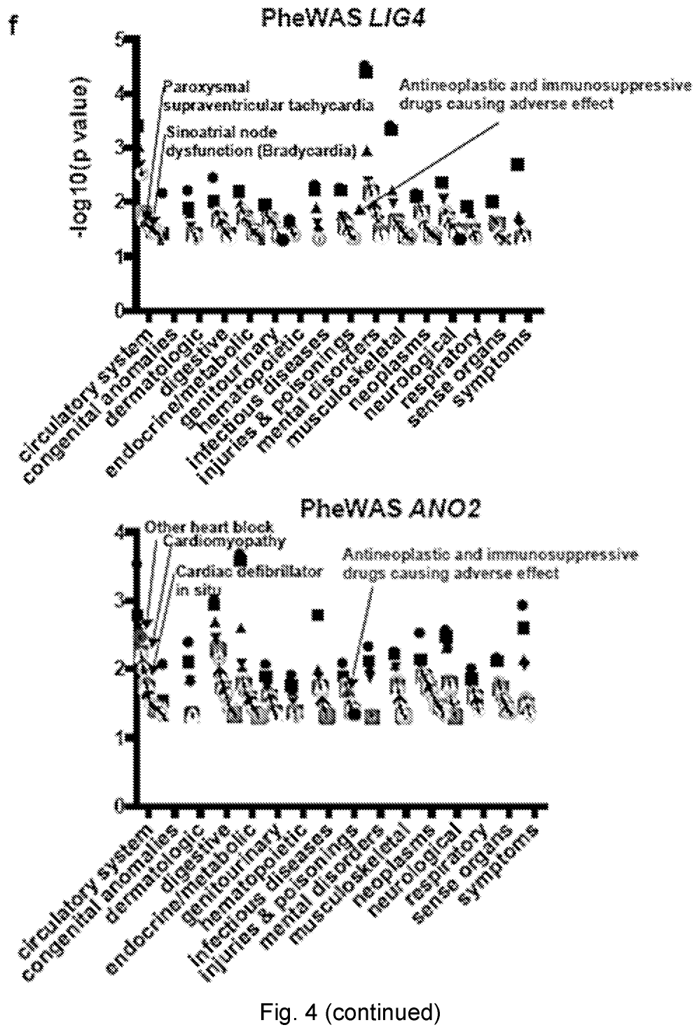

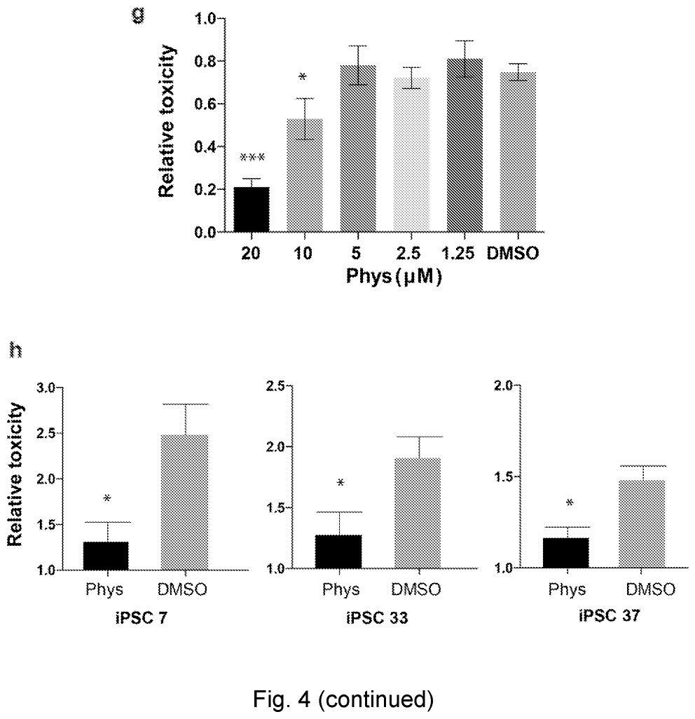

With an effective protocol to derive human SAN-like cells, we next established a platform to model sensitivity of pacemaker cells to drug toxicity. Some of the most common forms of arrhythmias are iatrogenic and occur as a consequence of chemotherapy treatment, requiring a permanent pacemaker replacement in severe cases. We focused on doxorubicin (DOXO) because it has well known adverse effects on the heart, thereby limiting its application for management of breast cancer and other tumors. Importantly, individual patients display different levels of sensitivity to this drug, indicating a contribution of genetic variation to this toxicity. This can be tested by modeling drug toxicity in human pacemaker cells derived from various genetic backgrounds (FIG. 9a). SHOX2:GFP⁺ hPSC-SAN cells or SHOX2:GFP⁻; MHC6:mCherry⁺ cardiomyocytes were differentiated to day 35, treated with different doses of DOXO for 3 days, and monitored for the percentage of cleaved caspase3⁺ cells. A drug dose was discovered (0.16 μM) at which the pacemaker-like cells showed significant cytotoxicity, while the cardiomyocytes displayed no significant increase in cell death (FIG. 4a, FIG. 9b).

To identify genetic variation that associates with the sensitivity of pacemaker cells to DOXO-induced toxicity, we performed an unbiased screen using a library of hPSC-SAN cells differentiated from 48 distinct iPSC lines. Cells differentiated for 35 days were treated with 0.16 μM DOXO for 3 days. Due to the lack of a robust antibody to SHOX2, an antibody recognizing ISL1 that is co-expressed with SHOX2:GFP (FIG. 2a), was used to identify hPSC-SAN cells that co-stain for cleaved caspase-3, as an assay for cytotoxicity. The hPSC-SAN cells derived from all of the lines showed at least some increase in the percentage of cleaved caspase3⁺-ISL1⁻ cells following treatment with DOXO. We identified 14 “high-sensitive” iPSC lines that generate hPSC-SAN cells displaying DOXO-induced apoptosis rates greater than 4-fold compared to DMSO-treated cells (FIG. 4b). To test if this platform can be applied toward in vitro pharmacogenomics, the iPSC clones were sequenced at several genomic regions harboring known SNPs associated with increased DOXO-mediated cardiotoxicity, including rs1056892, rs2229774 and rs885004 (in or near the genes CBR3, RARG and SLC28A3, respectively). 10 out of the 14 high-sensitive iPSC lines harbored one of these 3 SNPs (FIG. 4c). All 4 of the lines carrying the risk allele of rs1056892 generated hPSC-SAN cells with more than 4-fold increased toxicity. This is consistent with findings that CBR3 is expressed in heart tissue and functions for drug metabolism.

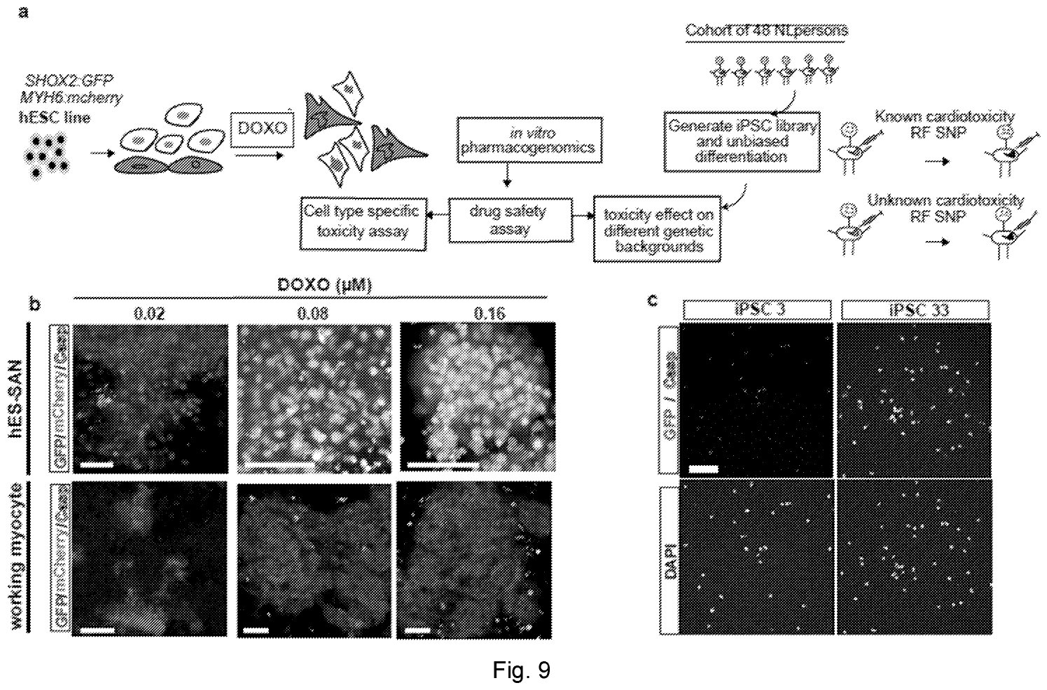

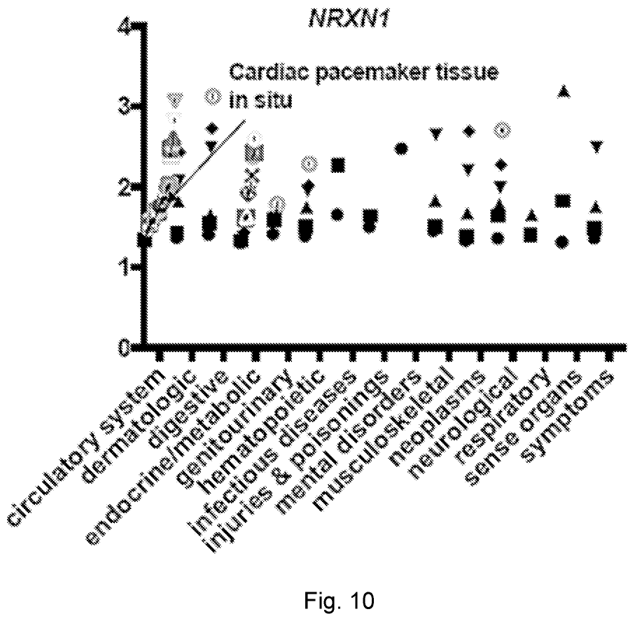

The remaining lines, including several high-sensitive lines, had no known risk alleles associated with DOXO-induced cardiotoxicity. Therefore, we sought to identify novel genetic variants associated with chemo-toxicity of SAN tissue. Genomic DNA from each of the iPSC lines was hybridized to Illumina arrays to genotype SNPs across the human genome. The SNP data was then analyzed for associations with cell toxicity (FIG. 4d). A total of 65 genetic variants showing suggestive associations (p<10⁵) with pacemaker toxicity were selected for further analysis, to determine whether the in vitro platform can successfully identify genetic variants that predict outcome in human patients. For this purpose, we studied a cohort of 384 patients who underwent chemotherapy. 52 of the patients presented with early-onset atrial fibrillation (AF) and 16 required placement of artificial permanent pacemakers (ppm). SNPs from three candidate loci identified in the in vitro hPSC-SAN-toxicity study were nominally associated (p<0.025; 0.05/2 phenotypes) with AF (1 locus, rs9559211, LIG4) and ppm (2 loci, rs7314566, ANO2 and rs17267852, NRXN1). (FIG. 4e). Interestingly, the function of LIG4 is tied to DNA Topoisomerase II (TOPII), which is a direct target of anthracyclines. Cells deficient in LIG4 and patients with mutations in LIG4 are hyper-sensitive to TOPII targeting drugs. Using unbiased phenome-wide scanning (PheWAS) in an electronic health record (EHR) cohort, we identified associations (p<0.05) between variants in genes from candidate loci and either neoplasm-related adverse effects or symptoms related to conduction system pathology (FIG. 4f, FIG. 10).

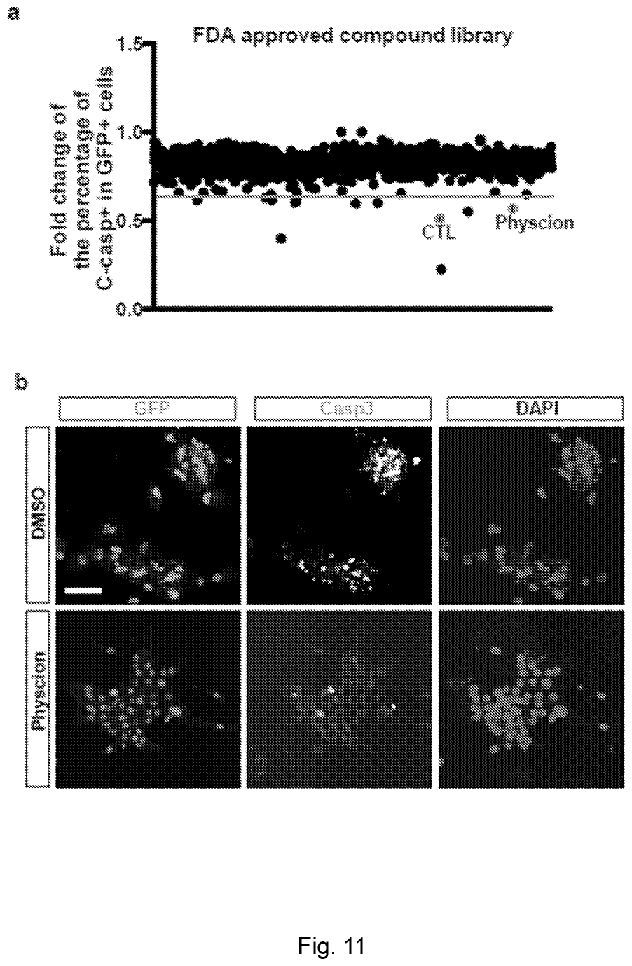

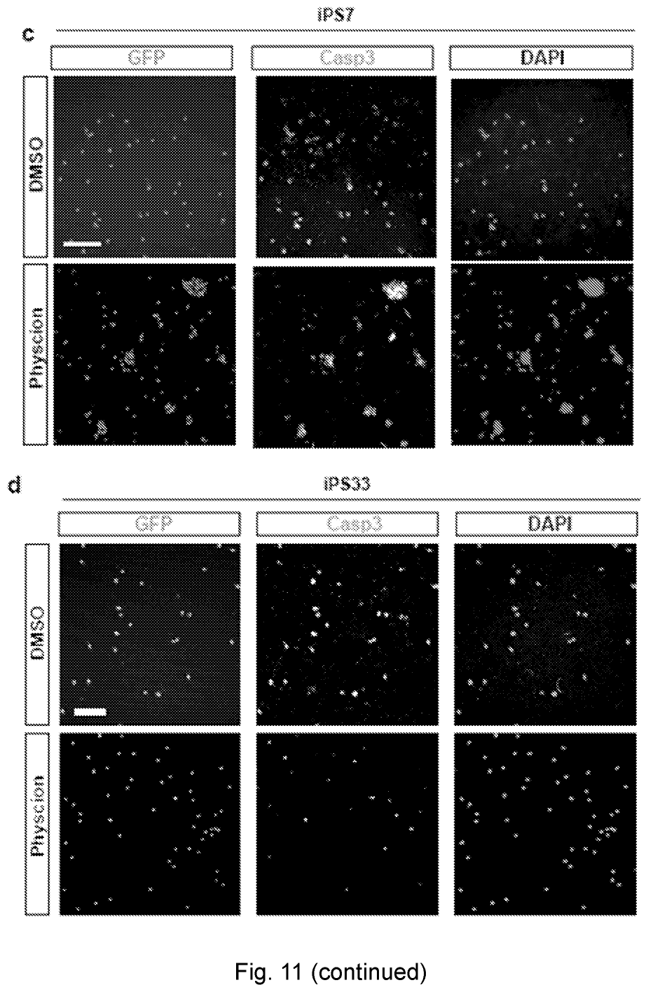

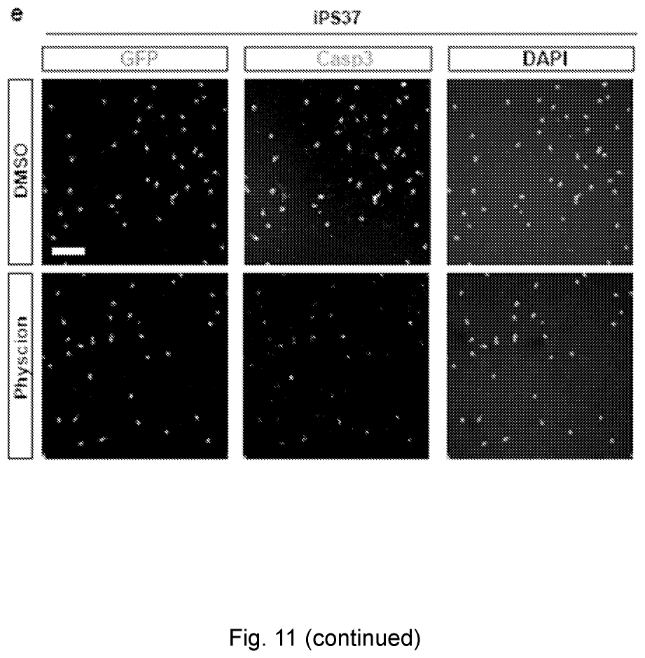

These findings demonstrate that hPSC-SAN cells can be used to model DOXO-induced cytotoxicity and discover novel genetic variants that are associated with arrhythmia phenotypes. With this validated platform, we carried out a small molecule screen to identify compounds capable of rescuing the toxicity effects observed with DOXO-treatment of hPSC-SAN cells. 1472 compounds were tested consisting of FDA-approved drugs and natural products. Hit targets were selected based on a reduced percent ratio of GFP⁺ cleaved caspase-3⁺/GFP⁺ cells (FIG. 11a). The compounds that decreased the cell death rate by more than two-fold of the standard deviation below the average of DMSO-treated samples were selected as primary hits. Physcion, a naturally occurring anthraquinone and an aldehyde reductase inhibitor, was further validated (FIG. 11b). Aldehyde reductases have been shown to convert DOXO into alcohol metabolites in the cardiomyocyte cytoplasm that results in significant cardiac toxicity. Physcion was effective at a dose of 10-20 μM. To determine whether this drug can protect the high-sensitive iPSC line derivatives, three of the most sensitive iPSC lines were differentiated to SAN-like cells and treated with DOXO in the presence or absence of 10 μM Physcion. Immunostaining revealed that inclusion of the compound led to a significant decrease in toxicity levels for hPSC-SAN cells derived from all three lines (FIG. 4h).

Methods:

Culture of hESC lines. hESC line H9 and the derivative (SHOX2:GFP; MYH6:mCherry) line, and 48 iPSC clones were grown on matrigel-coated plates and maintained in mTeSR medium (Stem Cell Technologies).

Pacemaker and cardiomyocyte induction. Differentiation was initiated 72 hours after plating when the culture was approximately 80% confluent. Step 1: Cells were differentiated with 1.5 μM CHIR99021 (CHIR, Stem-RD, Ch), 20 ng/mL BMP4 (B) and 20 ng/mL Activin A (AA) in RPMI (Cellgro) supplemented with B27 minus insulin, 2 mM GlutaMAX, 1× NEAA and 1× Pen/Strep for 3 days (RB27-INS). Step 2: For cardiomyocyte differentiation, cells were treated for an additional 3 days with 5 μM XAV939 (Tocris). For hPSC-SAN differentiation, Step 1 was followed by addition of 0.1 μM cucurbitacin (Cu, Sigma Aldrich), 1 μM retinoic acid (RA), 5 μM SU5402 (SU, Fi, Tocris) in RB27-INS from day 3-6. 5 μM XAV939 (Wi) was added from day 5-6. From day 6 onward, both cardiomyocyte and hPSC-SAN differentiation were carried out in RPMI supplemented with B27, 2 mM GlutaMAX, 1× NEAA and 1× Pen/Strep (RB27+INS). Step 3: hPSC-SAN differentiation included additional treatment with 5 μM Tyrphostin AG 490 (Sigma Aldrich) from day 6-9 in RB27+INS. The HDAC inhibitor, chidamide, was added to the differentiation cocktail from day 7-9 with a final concentration 5 μM. Variations are shown in FIG. 1a.

Gene expression analysis. Total RNA was isolated using the Qiagen RNeasy mini kit according to manufacturer instructions. cDNA synthesis was performed using TAKARA PrimeScript 1^{st} strand cDNA synthesis kit. qRT-PCR reactions were generated using Roche SYBR green PCR mix (Roche). Each data point represents at minimum 3 independent biological replicates. Primary antibodies and working dilutions are listed in Supplementary Table 1.

FACS and Immuno-fluorescence. For FACS analysis, the cells were dissociated with Accutase (Innovative Cell Technologies) for 15 min at 37° C. and fixed and permeabilized with the FOXP3 Fixation/Permeabilization set (eBiosciences). Subsequently, they were washed, blocked and permeabilized using FOXP3 permeabilization buffer (eBiosciences) according to the manufacturer's protocol. The cells were stained with primary (overnight 4 degrees) and secondary (45 mins, room temperature) antibodies and analyzed using a flow cytometer. For immunofluorescence, cells were fixed with 4% PFA (eBiosciences) for 20 min at room temperature. Next, cells were blocked and permeabilized in 5% horse serum (Invitrogen), 0.3% Triton X in PBS for one hour at room temperature, followed by primary antibody incubation overnight. After several rounds of washing, secondary antibodies were added for 1 hour at room temperature. Nuclei were stained with DAPI. Primary antibodies and working dilutions are listed in Supplementary Table 1.

Generation of the SHOX2:NLS-eGFP;MYH6:mCherry dual hESC reporter line. The αMHC:mCherry reporter is described in a complementary manuscript. For targeting the SHOX2 locus, sgRNA sequences were designed using the website http://crispr.mit.edu/and targeted the sequence: 5′-GGCGTTGGCGTCACAGACCC-3′ (SEQ ID NO:37). sgRNA was cloned into the PX459 vector (Addgene, plasmid #42230). For constructing the SHOX2:NLS-eGFP donor plasmid, the NLS-eGFP (from Tol2kit plasmid), codon optimized P2A, and left and right homology arms (from H9 genomic DNA) were PCR amplified and assembled using the In-Fusion kit (Clontech) and then cloned into the zero-blunt plasmid (Thermo Fischer). H9 hESCs were disassociated into single cells with Accutase for 5-7 min. Cells were electroporated using human stem cell nucleofector kit2 (Lonza, #VPH-5022) according to the manufacturer guidelines. Briefly, 1 million cells were resuspended in 100 μL nucleofector mix to which was added 2 μg CRISPR targeting plasmid and 4 μg donor plasmid. Cells were re-plated on matrigel coated plates with ROCK inhibitor. Two days after electroporation cells were treated with puromycin (0.5 μg/mL) for two days. About two weeks after electroporation individual colonies were picked and expanded for PCR genotyping and sequencing for the reporter lines. The sequences for all primers used for generating the donor plasmids are listed in Supplementary Table 2.

RNA Sequencing and single cell Drop-seq Analysis. For RNA sequencing, total RNA was isolated using the Agilent nano kit according to manufacturer instructions. The quality of RNA samples was examined using an Agilent bioanalyzer. cDNA libraries were generated using TruSeq RNA Sample Preparation (Illumina). Each library was sequenced using a single-read 50 bp in HiSeq4000 (Illumina). Gene expression levels were analyzed with TopHat and Cufflinks by the Weill Cornell Genomic Core facility. Raw data were normalized to that from SHOX2:GFP negative cells to define fold-change. Gene lists were filtered based on expression level differences ≥4 or ≤−4. Murine sinoatrial and atrial tissues were obtained from a published database (GSE65658). False discovery rate (FDR) q values <0.25 or nominal (NOM) p values <0.05 were considered significant. The expression data were normalized for each gene by subtracting their mean across samples and then dividing by their standard deviation. The normalization was performed separately on three sets of samples (D25 samples, D40 samples and public samples) to remove batch effects introduced by experiments. The heatmap plot was generated using heatmap.2 in the R “gplots” package.

Single cell sequencing. Cells were differentiated into hPSC-SAN or cardiomyocyte lineages. hPSC-SAN cells were sorted based on eGFP, while hPSC-V cells were sorted based on mCherry. Single-cells were captured using a Drop-Seq system. The two aqueous suspensions—the single-cell suspension and the barcoded primer beads suspended in a lysis buffer, were loaded into 3 mL plastic syringes (BD) respectively. Droplet generation oil (BioRad) was loaded into a 10 mL plastic syringe (BD). The three syringes were connected to a 125 μm co-flow device by 0.38 mm inner-diameter polyethylene tubing (Scientific Commodities, Inc.), and injected using syringe pumps (KD Scientific), resulting in ˜125 μm emulsion drops with a volume of ˜1 nL each. The flow was monitored under an inverted microscope. Droplets were collected in 50 mL falcon tubes; the collection tube was changed out after every 1 mL of combined aqueous flow volume. During droplet generation, the beads were kept in suspension by continuous, gentle magnetic stirring (V&P Scientific). The uniformity in droplet size and the occupancy of beads were evaluated by observing aliquots of droplets under the microscope. The oil from the bottom of each aliquot of droplets was removed after which 30 mL 6×SSC (Life Technologies) at room temperature was added. Collected droplets were broken by adding perfluorooctanol in 30 mL 6×SSC to destabilize the oil-water interface and the microparticles were disrupted. RNA-hybridized beads were extracted. The extracted beads were then washed and resuspended in a reverse transcriptase mix, followed by a treatment with exonuclease I to remove unhybridized, non-extended primers. The beads were then washed, counted, and aliquoted into PCR tubes for PCR amplification. 15-18 cycles of PCR amplification were applied to reach cDNA concentration at 400-1000 μg/μL after pre-PCR test step. The final PCR products were purified and pooled, and the amplified cDNA quantified on an Agilent 2100 BioAnalyzer High Sensitivity Chip. The cDNA was fragmented and amplified for sequencing with the Nextera XT DNA sample prep kit (Illumina) using custom primers. The libraries were purified, quantified, and sequenced on the Illumina NextSeq 500 at >50,000 reads/cell.

After quality assessment of the sequencing with FastQC v0.11.3, all single-cell RNA-seq samples were processed according to the Drop-seq Computational Protocol with the Drop-seq Toolkit v1.2 created by the McCarroll Lab. Sequences were first tagged with their corresponding cell and molecular barcodes (unique molecular identifiers, UMI) using dropseq TagBamWithReadSequenceExtended. Cell and molecular barcodes were required to have a minimum base quality of 10 across their lengths, otherwise they were discarded. SMART adapter sequences were trimmed from the 5′ end of each read whenever there were at least five continuous bases exactly matching the SMART adapter (dropseq TrimStartingSequence). Putative poly(A) tails were trimmed from the 3′ end whenever there were stretches of at least 6 adenosines with zero mismatches (dropseq PolyATrimmer). The UMI-tagged and trimmed reads were then aligned with default parameters to the human reference genome (GRCh38) using STAR (v2.4.2a). The final file containing alignment information plus the cell and UMI tags was generated with Picard MergeBamAlignment and Picard MergeBamAlignment. Sequences that mapped to more than one locus were excluded from further analysis by filtering reads with a mapping quality lower than 10.

To determine the numbers of detected transcripts per gene and cell, the overlaps of the aligned reads with gene annotation (Ensembl release 76) were counted and summarized using dropseq TagReadWithGeneExon and dropseq DigitalExpression. Individual gene expression matrices from each sample were combined in R to create a single gene count matrix for all cells and genes.

The count matrix was read into R; basic quality metrics such as the distributions of the numbers of gene counts per cell and gene were determined using in-house scripts and the scater package. We excluded cells with fewer than 100 UMI or fewer than 200 genes per cell, and more than 15% of the UMI mapping to mitochondrial genes. This resulted in 1,571 GFP-negative and 2,057 GFP-positive cells (representing about equal numbers per technical replicate). After filtering the cells, we excluded genes that were expressed in fewer than 5 cells per condition. Additionally, genes had to be covered by at least 10 UMI across all cells of the same condition. 13,448 genes met these filtering criteria and were used for downstream analyses. The UMI counts were adjusted for the differences in the individual cells' coverage by dividing every gene count by the total number of UMI counts of the respective cell (library size) and multiplying with the median of all library sizes. These scaled UMI counts were then further de-noised using the MAGIC algorithm. All expression values shown in the manuscript are log 10-transformed values (with an offset of 0.01 to avoid taking the log of zeros) after applying MAGIC. t-SNE was performed with the Rtsne package. Hierarchical clustering was applied to all cells and a subset of genes and visualized with the pheatmap function. Genes and cells were clustered with the base R hclust function using Euclidean distances and the agglomeration function “complete”.

Calcium imaging. hESC-derived cells were plated on ibidi plates coated with gelatin. The cells were loaded with 2 μM Fluo-4 AM dissolved in 1:1 (v/v) of 20% Pluronic®-F127 and DMSO with stock concentration of 1 mM for 45 min at RT in Tyrode solution consisting of (mM): 140 NaCl, 5.4 KCl, 1 MgCl2, 1.8 CaCl2), 10 glucose and 10 HEPES at pH 7.4. Calcium transients of hESC-derived beating cardiac clusters were recorded on a heated stage using a confocal scanning microscope (Zeiss LSM 710) at intervals of 200 ms (5 frames per second). They were then quantified as the background subtracted fluorescence intensity changes normalized to the background subtracted baseline fluorescence using MetaXpress software.

Cellular electrophysiology and characterization. Spontaneous action potentials were recorded over 10 seconds with the perforated patch clamp technique using an AM-Systems (WA, USA) model 2400 amplifier in current-clamp mode and the software platform Real-Time eXperiment Interface (RTXI; rtxi.org). Cells were superfused at 35° C. with a Tyrode's solution containing in mM: 137 NaCl, 5.4 KCl, 2 CaCl2), 1 MgSO4, 10 HEPES, and 10 glucose at pH 7.35 (NaOH). Whole cell access was achieved using 480 μg/mL Amphotericin-B (Sigma-Aldrich, MO, USA) in a pipette solution also containing in mM: 5 NaCl, 20 KCl, 120 K-aspartate, and 10 HEPES at pH 7.2 (KOH). A liquid junction potential of 14 mV was corrected. Pipettes were pulled from 1.5 mm capillary tubes (AM-Systems, WA, USA) to a resistance between 2.5-3.5 MΩ. Cells were characterized as nodal-like if they exhibited spontaneity and a slow upstroke (dV/dtₘₐₓ<30 V/s).

SNP array and GWAS analysis. High-throughput genotyping of 906,600 SNPs was performed on Affymetrix Genome-Wide Human SNP Array 6.0 (Santa Clara, Calif., USA) at Core Facility of Albert Einstein College of Medicine following the manufacturer's instructions (Affymetrix, Inc., Santa Clara, Calif., USA). Genotyping was performed using the default parameters in the Birdseed v2 algorithm of Genotyping Console (GTC) 4.2 software (Affymetrix). As a quality control for the genotyping, Contrast QC values were calculated as implemented in the GTC 4.2, and samples used passed the recommended values. Genome annotations applied in data analysis referred to the human reference assembly GRCh37/hg19 as provided by the Affymetrix annotation file. In this study, SNPs with low minor allele frequencies (<0.05), low call rates (<95%), and inconsistent genotype frequencies with Hardy-Weinberg equilibrium (P<1.0×10⁻⁵) were excluded. In addition, allosome SNPs were not analyzed. After quality control, a total of 691,822 common SNPs were included in the association analysis. An iterative procedure was used to simultaneously estimate principal components (PCs) reflecting population structure. Linear regression was applied to examine associations of SNPs and phenotypes under an additive model adjusted for sex and two principal components. Analyses were performed using R (version 3.3.2 R Foundation).

Association with anthracycline-association arrhythmia phenotypes in humans. Variants identified in cell-based screens were tested for association in a human cohort of subjects (N=384) treated with anthracyline-based chemotherapy. The cohort was developed from BioVU, the Vanderbilt University biobank linking DNA samples (and any related genotype data) extracted from blood leftover after routine clinical care at Vanderbilt University Medical Center to a de-identified version of the electronic health record (EHR) intended to support research. Details of the cohort as well as the development and implementation of BioVU have been described (PMID: 28542097, 18500243). Subjects with atrial fibrillation (AF) or pacemaker implantation after chemotherapy initiation were identified using key word searches and automated queries of billing and procedure codes. Subjects were genotyped in the Vanderbilt Technologies for Advanced Genomics (VANTAGE) Core using the Illumina Omni 1-Quad platform using standard quality control filters at the sample and variant level. Genotypes were imputed to 1000 Genomes Phase 1 version 3 (Apr. 19, 2012) using reference panels from all 1000 Genomes populations. Variants were tested for association with AF and pacemaker implantation separately using multiple logistic regression, implemented in PLINK, adjusted for age, race, gender. As this was a hypothesis-generating “replication” from our cell-based “discovery” analyses, we chose a significant threshold of 0.025 (0.05/2 phenotypes).

PheWAS analysis. To assess whether genes from identified loci have broader phenotypic relevance (i.e., outside patients treated with chemotherapy) we conducted phenome-wide association studies (PheWAS) in subjects with available genotype data [25,539 on the Illumina HumanExome BeadChip v.1.0 (‘exome chip’) and 13,331 on the Illumina Multi-Ethnic Genotyping Array (MEGA) platform] generated as part of ongoing research efforts. All subjects were of European decent and subject overlap between the two platforms was modest (2,729). The PheWAS method utilized a validated medical phenome of hierarchically grouped International Classification of Disease (ICD-9) billing codes into ˜1,800 phenotypes (‘phecodes’), each with defined control groups. Logistic regression was used to serially test for association between genetic variants in candidate genes and each phecode phenotype. All analyses were adjusted for age and gender. As these were exploratory analyses, we chose a liberal threshold of 0.05 for evidence of nominal association with each phenotype.

Chemical screen. The high-throughput chemical screening was performed by Prestwick FDA approved and natural products libraries. The 30 day old hES-SAN cells derived from the dual-reporter line were replated into matrigel coated 384-well plates. The DOXO was added at final concentration as 0.16 μM. At the same time, individual compound from chemical library was added at final concentration as 10 μM and maintained for 3 days. The plates were fixed and stained with primary antibodies against GFP and Caspase-3. The compounds that decreased the cell death rate by more than two-fold of the standard deviation below the average of DMSO-treated samples were selected as primary hits. For validation, the cells treated with varying doses of the hit compound and a dose curve was generated. Finally, the sensitive iPS lines were treated with 10 μM physcion and DOXO to validate the efficacy on different cell lines and stained with ISL1 and Caspase-3 for scoring.

Data availability statements. The datasets generated during and analyzed during the current study are available in the GEO repository, which will be available to public upon publication. The reviewer link will be sent to editor once GEO number is assigned.

TABLE 1
(List of antibodies)

| Antigen | Source | Dilution |
|---|---|---|
| ISL1 | DSHB 39.4D5 | 1:400 |
| GFP | Abeam ab13970 | 1:1000 |
| HCN4 | Abeam ab85023 | 1:400 |
| TBX5 | Ptg 13178-1-AP | 1:400 |
| NKX2.5 | Ptg 13921-1-AP | 1:400 |
| Cav3.1 | Alomone ACC-021 | 1:400 |
| Cav1.3 | Alomone ACC-005 | 1:400 |
| Cx30.2 | Atlas antibodies HPA015024 | 1:400 |
| Synapsin | Enzo ADI-905-782-100 | 1:1000 |

TABLE 2
(List of primers)

| Gene | Forward | Reverse |
|---|---|---|
| hNPPA | TCGGAGCCTGCGGAGAT | GTCCTCCCTGGCTGTTATCT |
| | (SEQ ID NO: 1) | TC (SEQ ID NO: 2) |
| hTBX3 | AAAGGAGAATGGGACCTCTG | CGCTGGGACATAAATCTTTG |
| | (SEQ ID NO: 3) | AG (SEQ ID NO: 4) |
| hTBX18 | TTGCTAAAGGCTTCCGAGAC | AGGTGGAGGAACTTGCATTG |
| | (SEQ ID NO: 5) | (SEQ ID NO: 6) |
| hTBX5 | ACAGCGTTCTGCACTCACGT | CATGTCATCACTGCCCCGAA |
| | (SEQ ID NO: 7) | AT (SEQ ID NO: 8) |
| hNKX2.5 | TCTATCCACGTGCCTACAGC | GTTGTCCGCCTCTGTCTTCT |
| | (SEQ ID NO: 9) | (SEQ ID NO: 10) |
| hISL1 | TGATGAAGCAACTCCAGCAG | GGACTGGCTACCATGCTGTT |
| | (SEQ ID NO: 11) | (SEQ ID NO: 12) |
| hHCN4 | GTCTTTGTTTGGGGCAAGAG | GATTGGATGGCAGTTTGGAG |
| | (SEQ ID NO: 13) | (SEQ ID NO: 14) |
| hSHOX2 | AATCAAGCAGAGGCGAAGTC | TCTCGTCAAAAAGCCTCTCC |
| | (SEQ ID NO: 15) | (SEQ ID NO: 16) |
| hMYH6 | GTGCTGGCCCTTCAACTAC | GCAGCTTCTCCACCTTAGC |
| | (SEQ ID NO: 17) | (SEQ ID NO: 18) |
| rs1056892 | GTGTTTAAGGGCTTTTGAAA | CATTCCACCAACATTTATTA |
| (CBR3) | ACTGC (SEQ ID NO: | AGCAGC (SEQ ID NO: |
| | 19) | 20) |
| rs2229774 | ACAGTCCAGGCTTAGAAGGA | ACAGCATTAGGCACCAAGGG |
| (RARG) | TGTC (SEQ ID NO: 21) | TAGGAG (SEQ ID NO: |
| | | 22) |
| rs885004 | GTCCATGCTGTACTACCTGG | GTAGCGGTTCTCCTCTCTAG |
| (SLC28A3) | GACT (SEQ ID NO: 23) | CCTG (SEQ ID NO: 24) |
| mTbx3 | CCACCTCCAACAACACGTTC | TAAGGAAACAGGCTCCCGAA |
| | T (SEQ ID NO: 25) | (SEQ ID NO: 26) |
| mTbx18 | TGTCCCCCATCAAGCCTGTT | ATGGCCTCCAGAATGCGTAT |
| | (SEQ ID NO: 27) | G (SEQ ID NO: 28) |
| mShox2 | ACCAATTTTACCCTGGAACA | TCGATTTTGAAACCAAACCT |
| | AC (SEQ ID NO: 29) | G (SEQ ID NO: 30) |
| mGapdh | CTAACATCAAATGGGGTGAG | CGGAGATGATGACCCTTTTG |
| | G (SEQ ID NO: 31) | (SEQ ID NO: 32) |
| SHOX2::GFP | ACCCGCAGTCTTTCCAATAT | AAGTCGTGCTGCTTCATGTG |
| genotyping, | ACCATTTCAGCCCA (SEQ | GTCGGGGT (SEQ ID NO: |
| left | ID NO: 33) | 34) (inside GFP) |
| homology | | |
| arm | | |
| SHOX2::GFP | TGGCGGAATGGTGAGCAAGG | ATGGGAGATAAGGGGGTGGG |
| genotyping, | GCGA (SEQ ID NO: 35) | AGGAGA (SEQ ID NO: |
| right | (inside GFP) | 36) |
| homology | | |
| arm | | |

### Example 2

This example provides further studies on characterization of the SAN-like cells generated by the present methods and the use of these cells to identify cardiac toxicity reducing agents.

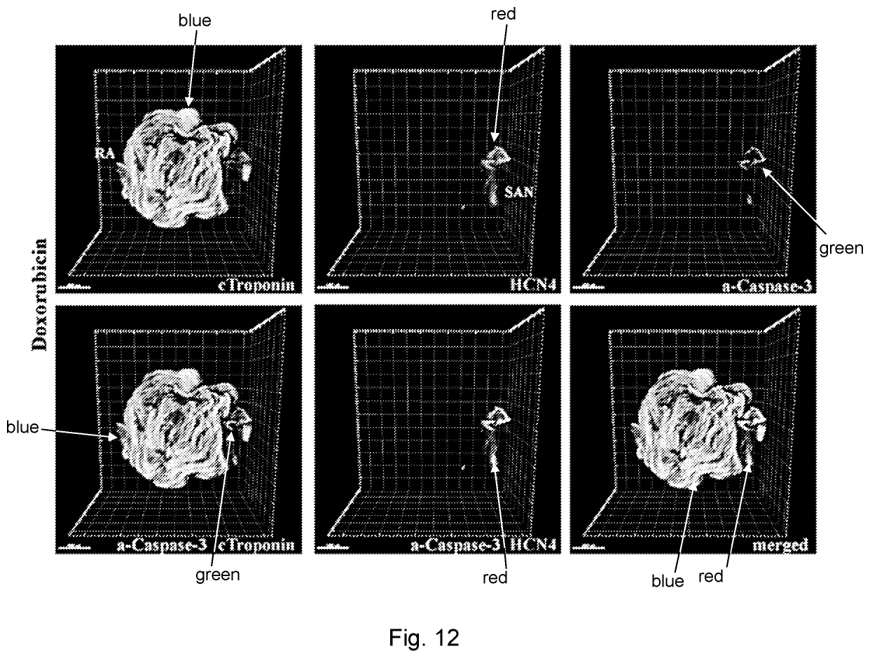

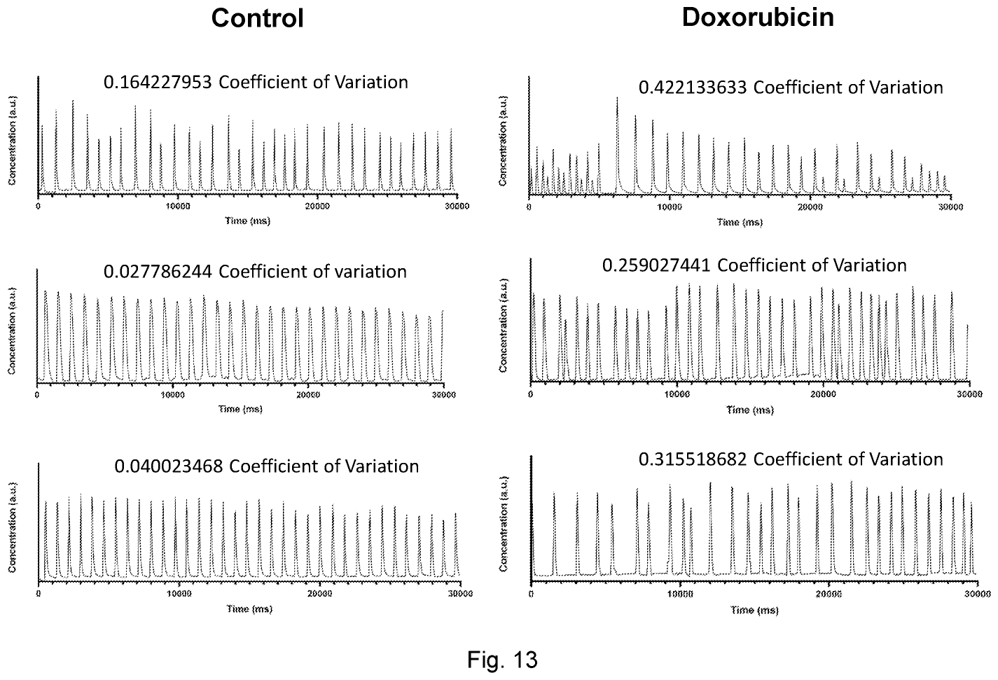

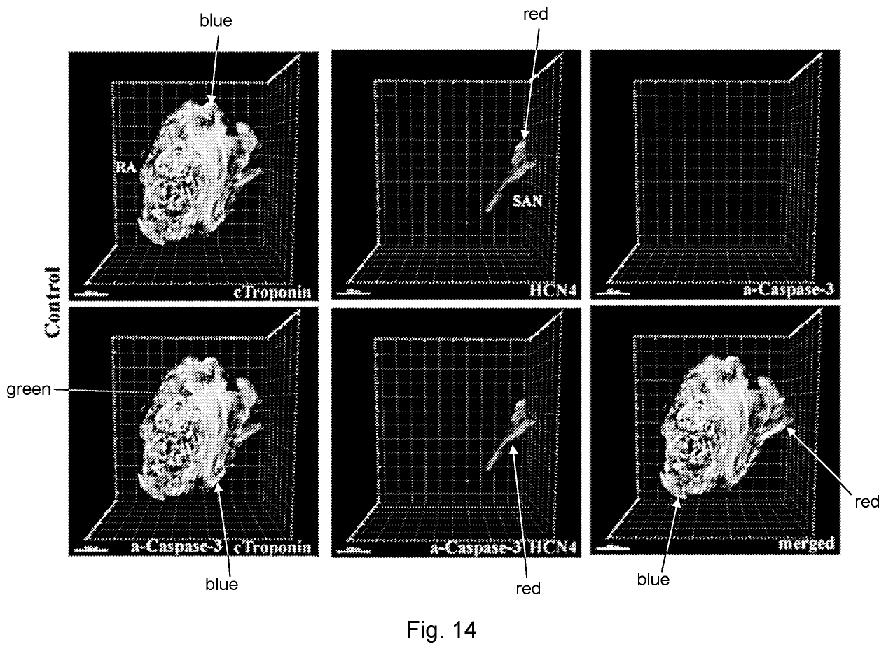

Biased pacemaker cell death has yet to be reported as a cardiotoxic side effect of Doxorubicin. To corroborate this in vitro discovery, we assayed Doxorubicin toxicity in vivo in mice. C57BL/6 mice were treated with 20 mg/kg Doxorubicin by intraperitoneal injection. 9 days post injection, the mice were euthanized and whole heart imaging of mouse heart revealed cell death localized to the HCN4+ SAN cells (FIG. 12). Consistent with putative SAN dysfunction, hearts of mice treated with Doxorubicin exhibited irregular contractions (FIG. 13). By contrast, hearts of control mice lacked cell death (FIG. 14) and exhibited rhythmic contractile activity (FIG. 13).

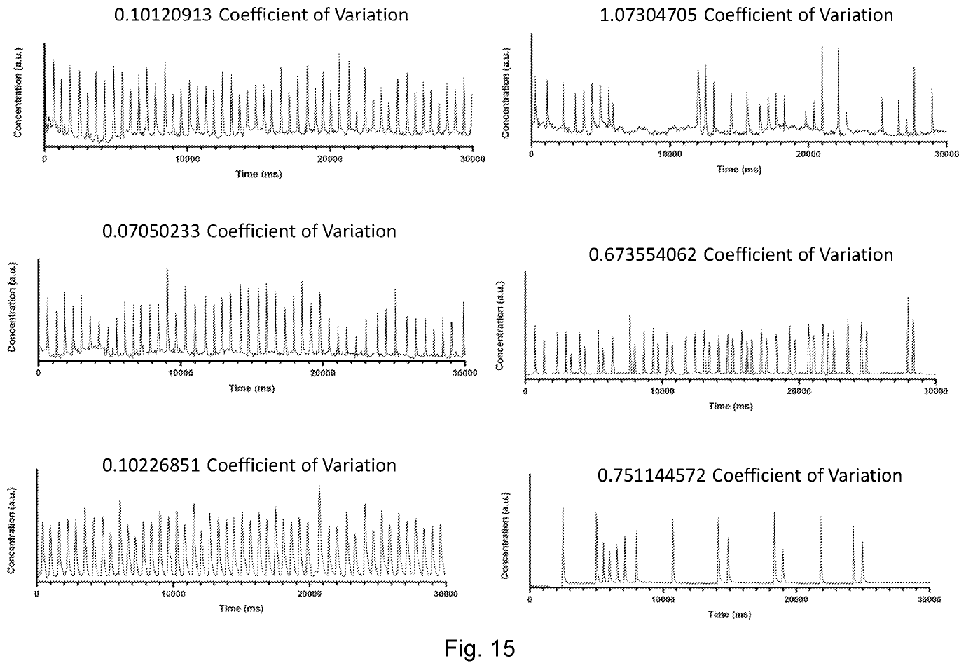

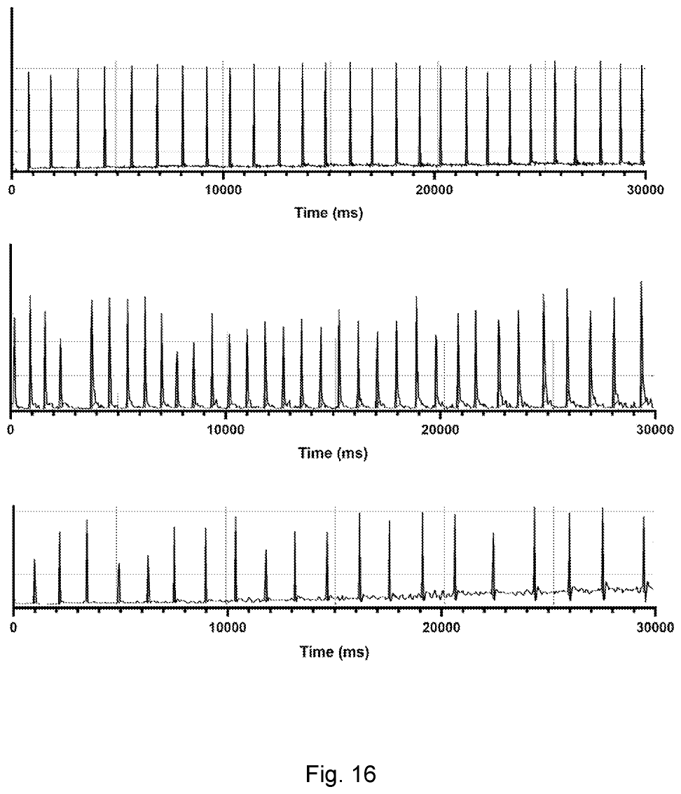

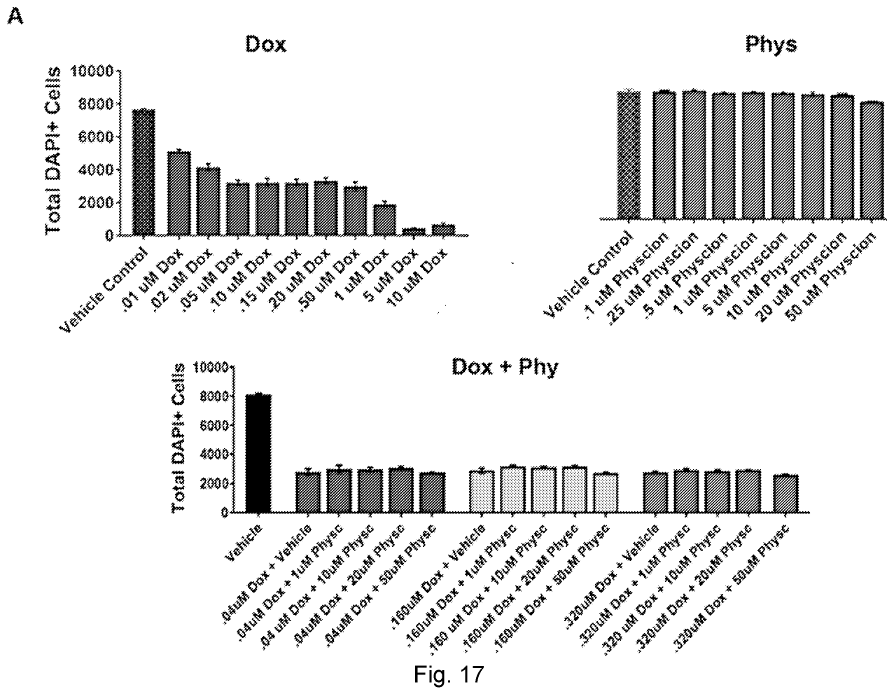

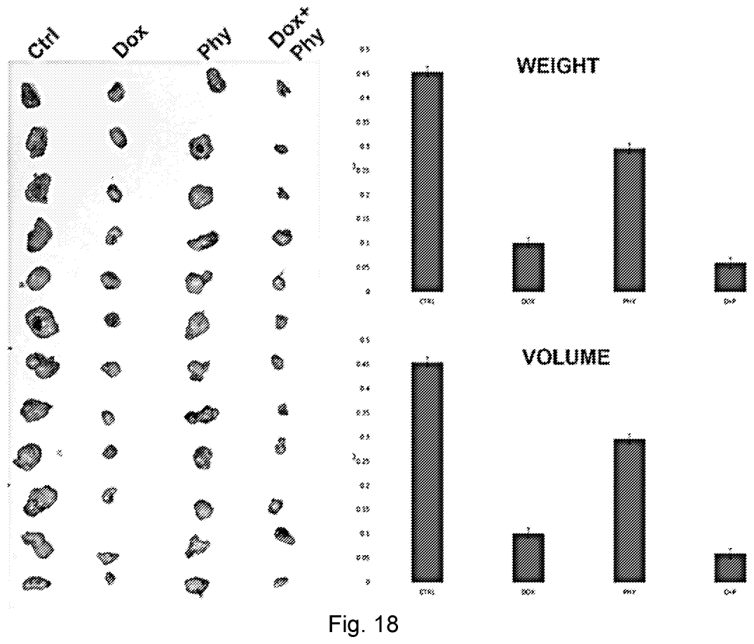

The restorative effect of Physcion identified in our drug screen was gauged in the bona fide SAN. doxorubicin toxicity in vivo is a multifactorial process that impedes the differentiation between correlative and causative factors. Thus, we used an ex vivo model to document the direct effect of Doxorubicin and Physcion on the murine SAN. Whole, intact atrial explants were incubated with 1 μM doxorubicin or 1 uM doxorubicin+20 μM Physcion for 3 hrs. Consistent with our in vivo doxorubicin treatment studies, control explants lacked cell death and had rhythmic contractions; while doxorubicin-treated explants exhibited localized SAN cell death and irregular contractility (FIG. 15). Strikingly, physcion blocks doxorubicin-induced SAN cell death and arrhythmic contractility of explants (FIG. 16). Importantly, Physcion did not block the chemotherapeutic efficacy of doxorubicin in cell culture (FIG. 17) or xenograft cancer models (FIG. 18). Taken together, these results reveal Physcion as adjuvant that can mitigate side effects of cancer treatment regimens using doxorubicin.

Although specific embodiments and examples are provided those skilled in the art will recognize that routine modifications to these embodiments can be made which are intended to be within the scope of the invention.

What is claimed is:

1. A method of generating a population of cells with sinoatrial node (SAN) characteristics from pluripotent stem cells comprising:
a) incubating pluripotent stem cells with a medium comprising a GSK inhibitor, and one or more members of the TGF-β superfamily of proteins for a sufficient period of time to generate pre-cardiac mesoderm cells, wherein the pre-cardiac mesoderm cells express FLK1 and PDGFR-alpha;
b) incubating the pre-cardiac mesoderm cells with a medium comprising retinoic acid pathway activator, a WNT inhibitor, a ALK inhibitor, and a member of the TGF-β superfamily of proteins and/or an EGFR inhibitor for a sufficient period of time to generate pacemaker progenitor cells, wherein the pacemaker progenitor cells express TBX3, TBX5, TBX18, SHOX2, HCN4, and ISL1; and
c) incubating the pacemaker progenitor cells with a medium comprising a GSK inhibitor, and an EGFR inhibitor and optionally, a HDAC inhibitor for a sufficient period of time to generate a population of SAN-like cells, wherein the SAN-like cells continue to express TBX3, TBX5, TBX18, SHOX2, HCN4, and ISL1 and can exhibit HCN-4 dependent funny current.

2. The method of claim 1, further comprising continuing to culture the SAN-like cells from step c) in a serum-free medium for a sufficient period of time to generate a population of SAN-like cells that express CAV3.1, CAV1.3 and Cx30.2.

3. The method of claim 1, wherein the method comprises:
a) incubating pluripotent stem cells with a medium comprising CHIR99021, and BMP-4 and/or Activin A for a sufficient period of time to generate pre-cardiac mesoderm cells;
b) incubating the pre-cardiac mesoderm cells with a medium comprising retinoic acid, XAV939, SB43152 and BMP4 and/or SU5402, and optionally, cucurbitacin for a sufficient period of time to generate pacemaker progenitor cells; and
c) incubating the pacemaker progenitor cells with a medium comprising CHIR99021 and Tyrphostin AG-490 for a first period of time, and then with a medium further comprising chidamide for a second period of time sufficient to generate a population of SAN-like cells.

4. The method of claim 1, wherein in step b, the medium further comprises a STAT3 inhibitor.

5. The method of claim 1, wherein the SAN-like cells exhibit electrophysiological properties of SAN cells.

6. The method of claim 5, wherein the electrophysiological properties exhibited by SAN-like cells comprise exhibition of HCN-4 dependent funny current.

7. The method of claim 2, wherein the method comprises:
a) incubating pluripotent stem cells with a medium comprising 0.5 to 3 μM CHIR99021, and 5-100 ng/ml BMP-4 and/or 5-50 ng/ml Activin A for a sufficient period of time to generate pre-cardiac mesoderm cells;
b) incubating the pre-cardiac mesoderm cells with a medium comprising 0.1 to 10 μM retinoic acid, 1-10 μM XAV939, 1-10 μM SB43152 and 5-100 ng/ml BMP4 and/or 0.1-10 μM SU5402, and optionally, 0.1 to 5 μM cucurbitacin for a sufficient period of time to generate pacemaker progenitor cells; and
c) incubating the pacemaker progenitor cells with a medium comprising 0.5 to 3.0 μM CHIR99021 and 1-10 μM Tyrphostin AG-490 for a first period of time, and then optionally, with a medium further comprising 0.1-10 μM chidamide for a second period of time sufficient to generate a population of cells with SAN-like cells.

8. The method of claim 1 further comprising prior to step a) transfecting the cells with a reporter for SHOX2 expression, and selecting for the SHOX2 expressing cells at the end of c).

9. The method of claim 1, wherein the pluripotent stem cells are embryonic pluripotent stem cells.

10. The method of claim 1 wherein the pluripotent stem cells are induced pluripotent stem cells.

11. The method of claim 1, wherein the pluripotent stem cells are human pluripotent stem cells.

12. The method of claim 7, wherein the SAN-like cells exhibit HCN4-dependent funny current.

13. A population of cells generated by the method of claim 1, wherein at least some cells in the population express SHOX2, TBX18, HCN4, CAV3.1, CAV1.3, and Cx30.2. and exhibit HCN-dependent funny current.

14. The population of cells in claim 13, wherein the at least some cells do not express Cx40.

15. The population of cells of claim 14, wherein at least 50%, at least 60%, at least 70%, or at least 90% of the cells express SHOX2, TBX18, HCN4, CAV3.1, CAV1.3, and Cx30.2. and exhibit HCN-dependent funny current.

16. A method of testing for candidates that can mitigate drug induced cardiac toxicity comprising:
a) exposing a population of cells comprising SAN-like cells obtained by the method of claim 1 to a candidate drug or a drug suspected of causing or known to cause cardiac toxicity;
b) identifying changes in the expression of one or more of SHOX2, TBX18, HCN4, CAV3.1, CAV1.3, and Cx30.2 and/or HCN4-dependent funny current due to the presence of the drug;
c) screening candidate cardiac toxicity mitigating agents by detecting mitigation of changes seen in step b) and identifying agents as being able to mitigate drug induced cardiac toxicity if the agent is able to mitigate the changes seen in step b).

17. The method of claim 16, wherein the drug is an anthracycline.

18. The method of claim 17, wherein the anthracycline is doxorubicin.

19. A method of preventing or reducing anthracycline-induced cardiotoxicity comprising administering to a subject who has been administered, who is being administered or who is to be administered an anthracycline, a cardiac toxicity reducing amount of physcion or a cardiac toxicity reducing derivative thereof.

20. The method of claim 19, wherein the physcion derivative has the structure: 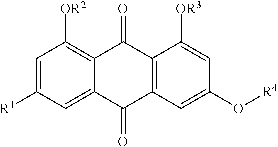
where R¹ and R⁴ are selected from substituted and unsubstituted alkyl groups and substituted and unsubstituted alkoxy groups, and R² and R³ are selected from hydrogen, substituted and unsubstituted alkyl groups, substituted and unsubstituted alkylcarbonyl groups, substituted and unsubstituted alkylsulfonyl groups, and substituted and unsubstituted alkylphosphonyl groups.

21. The method of claim 20, wherein the subject has one or more of the following SNPs:
a) rs1056892 in CBR3
b) rs2229774 in RARG
c) rs885004 in SLC28A3
d) rs9559211 in LIG4
e) rs7314566 in ANO2
f) rs17267852 in NRXN1
